SEQUENCE LISTING (30 sequences)

SEQ ID NO: 1 - DNA, 24 bp, Artificial Sequence
  Description of Artificial Sequence  oligomer probe
tacgagttgagaatcctgaatgcg

SEQ ID NO: 2 - DNA, 17 bp, Artificial Sequence
  Description of Artificial Sequence  probe
ctgagcacactcagtac

SEQ ID NO: 3 - DNA, 24 bp, Artificial Sequence
  Description of Artificial Sequence  oligomer complement of SEQ ID No 1 with single base mismatch at position 11
cgcattcaggcttctcaactcgta

SEQ ID NO: 4 - DNA, 24 bp, Artificial Sequence
  Description of Artificial Sequence  oligomer
tgcctacgaggaattccatagcta

SEQ ID NO: 5 - DNA, 10 bp, Artificial Sequence
  Description of Artificial Sequence  oligomer with rhodamine red label at 5′ end
caggaattcc

SEQ ID NO: 6 - DNA, 24 bp, Artificial Sequence
  Description of Artificial Sequence  oligomer with rhodamine red label at 5′ end
tagctatggaattcctcgtaggca

SEQ ID NO: 7 - DNA, 50 bp, Artificial Sequence
  Description of Artificial Sequence  oligomer with rhodamine red label at 5′ end
gaacctctgctcaacaagttccagattacaacttcaccaggttcaacaca

SEQ ID NO: 8 - DNA, 15 bp, Artificial Sequence
  Description of Artificial Sequence  oligomer with rhodamine red label at 5′ end
aggaattccatagct

SEQ ID NO: 9 - DNA, 50 bp, Artificial Sequence
  Description of Artificial Sequence  oligomer containing complement of SEQ ID NO 8 at positions 18-32
actaggcactgtacgccagctatggaattccttagctatgagatccttcg

SEQ ID NO: 10 - DNA, 50 bp, Artificial Sequence
  Description of Artificial Sequence  oligomer containing complement of SEQ ID NO 8 at positions 36-50
gttagctatgagatccttcgtaggcactgtacgccagctatggaattcct

SEQ ID NO: 11 - DNA, 50 bp, Artificial Sequence
  Description of Artificial Sequence  oligomer non-complementary to SEQ ID NO 8
tgtgttgaacctggtgaagttgtaatctggaacttgttgagcagaggttc

SEQ ID NO: 12 - DNA, 18 bp, Artificial Sequence
  Description of Artificial Sequence  forward primer
cctgggcattaaggttcc

SEQ ID NO: 13 - DNA, 20 bp, Artificial Sequence
  Description of Artificial Sequence  reverse primer
tgggattcttcggcttcttc

SEQ ID NO: 14 - DNA, 17 bp, Artificial Sequence
  Description of Artificial Sequence  probe
cctgtctaacaccacag

SEQ ID NO: 15 - DNA, 17 bp, Artificial Sequence
  Description of Artificial Sequence  probe
ccacagcttggtcagaa

SEQ ID NO: 16 - DNA, 17 bp, Artificial Sequence
  Description of Artificial Sequence  probe
accacacactgtctctc

SEQ ID NO: 17 - DNA, 15 bp, Artificial Sequence
  Description of Artificial Sequence  probe
cgggagatgcaggag

SEQ ID NO: 18 - DNA, 15 bp, Artificial Sequence
  Description of Artificial Sequence  probe
acggcaagctggagg

SEQ ID NO: 19 - DNA, 15 bp, Artificial Sequence
  Description of Artificial Sequence  probe
cttgccgtcaccgct

SEQ ID NO: 20 - DNA, 15 bp, Artificial Sequence
  Description of Artificial Sequence  probe
cagcggtgacggcaa

SEQ ID NO: 21 - RNA, 15 nt, Artificial Sequence
  Description of Artificial Sequence  probe
aggaauuccauagcu

SEQ ID NO: 22 - RNA, 15 nt, Artificial Sequence
  Description of Artificial Sequence complementary target of SEQ ID NO 21
agcuauggaauuccu

SEQ ID NO: 23 - RNA, 15 nt, Artificial Sequence
  Description of Artificial Sequence non-complementary target of SEQ ID NO 21
cgaucacgagaucga

SEQ ID NO: 24 - RNA, 15 nt, Artificial Sequence
  Description of Artificial Sequence  target of SEQ ID NO 21 containing single base mismatch at position 7
agcuauagaauuccu

SEQ ID NO: 25 - DNA, 15 bp, Artificial Sequence
  Description of Artificial Sequence  probe with rhodamine red label at 5′ end
aggaattccatagct

SEQ ID NO: 26 - DNA, 15 bp, Artificial Sequence
  Description of Artificial Sequence  target of SEQ ID NO 25
agctatggaattcct

SEQ ID NO: 27 - DNA, 50 bp, Artificial Sequence
  Description of Artificial Sequence complementary target of SEQ ID NO 25, with mismatch
actaggcactgtacgccagctatcgaattccttagctatgagatccttcg

SEQ ID NO: 28 - DNA, 50 bp, Artificial Sequence
  Description of Artificial Sequence complementary target of SEQ ID NO 25, with mismatch
gttagctatgagatccttcgtaggcactgtacgccagctatcgaattcct

SEQ ID NO: 29 - DNA, 15 bp, Artificial Sequence
  Description of Artificial Sequence  probe with rhodamine red label at 5′ end
ctgaatccaggagca

SEQ ID NO: 30 - DNA, 20 bp, Artificial Sequence
  Description of Artificial Sequence non-complementary target for SEQ ID NOS 6 and 28
atggcaactatacgcgctac

(19) United States
(12) Patent Application Publication
(10) Pub. No.: US 2005/0059042 A1
(43) Pub. Date: Mar. 17, 2005

(54) Colorimetric and fluorescent methods for sensing of oligonucleotides

(76) Inventors: Lewis J. Rothberg, Pittsford, NY (US); Huixiang Li, Rochester, NY (US)

Correspondence Address: Edwin V. Merkel;Nixon Peabody LLP, Clinton Square, P.O. Box 31051, Rochester, NY, 14603-1051, US

(21) Appl. No.: 10/847,233

(22) Filed: May 17, 2004

Related U.S. Application Data
(60) Provisional application No. 60/471,257, filed on May 16, 2003.
(60) Provisional application No. 60/552,793, filed on Mar. 12, 2004.

(51) Int. Cl. C12Q 1/68

(52) U.S. Cl. 435/6

(57) ABSTRACT

Methods and kits are provided for detecting the presence or absence of target nucleic acid sequences in a sample. The methods and kits involve the use of metal nanoparticles and the electrostatic interactions between the metal nanoparticles and nucleic acid molecules. The methods rely upon the differential interaction of ss-nucleic acids and ds-nucleic acids with the metal nanoparticles. A colorimetric detection approach utilizes the ability of ss-nucleic acids electrostatically associated with metal nanoparticles in a colloidal suspension to stabilize them against aggregation. A fluorescent approach involving tagged ss-oligonucleotide probes translates the differential adsorption of ss-nucleic acids on metal nanoparticles to differential quenching of a fluorescent tag on probes that have not hybridized with targets.

This application claims the benefit of U.S. Provisional Patent Application Ser. No. 60/471,257, filed May 16, 2003, and U.S. Provisional Patent Application Ser. No. 60/552,793, filed Mar. 12, 2004, each of which is hereby incorporated by reference in its entirety.

The present invention was made at least in part with funding received from the National Institutes of Health under grant AG18231. The U.S. government may retain certain rights in this invention.

### FIELD OF THE INVENTION

The present invention relates to hybridization-based nucleic acid detection procedures and materials for practicing the same.

### BACKGROUND OF THE INVENTION

Detection of specific oligonucleotide sequences is important for clinical diagnosis, biochemical and medical research, food and drug industry, and environmental monitoring, pathology and genetics (Primrose et al., Principles of Genome Analysis and Genomics, Blackwell Publishing, Malden, Mass., Third edition (2003); Hood et al., Nature 421:444-448 (2003); Rees, Science 296:698-700 (2002)). Present assays are dominated by chip based methodologies (Epstein et al., Analytica Chimica Acta 469:3-36 (2002); Chee et al., Science 274:610-614 (1996)) that have two principal disadvantages. First, target labeling is usually required. Second, hybridization to sterically constrained probes on surfaces is slow. Approaches such as sandwich assays (Elghanian et al., Science 277:1078-1081 (1997); Taton et al., Science 289:1757-1760 (2000); Cao et al., Science 297:1536-1540 (2002); Park et al., Science 295:1503-1506 (2002)), immobilized molecular beacons (Dubertret et al., Nat. Biotech. 19:365-370 (2001); Du et al., J. of Am. Chem. Soc. 125:4012-4013 (2003)), surface plasmon resonance (Brockman et al., Annual Review of Physical Chemistry 51:41-63 (2000)), porous silicon microcavity emission (Chan et al., Materials Science & Engineering C-Biomimetic and Supramolecular Systems 15:277-282 (2001)), and reflective interferometry (Lin et al., Science 278:840-843 (1997); Pan et al., Nano Lett. 3:811-814 (2003)) avoid the former problem, but still require complex surface attachment chemistry for probe immobilization and may suffer from slow response. In several of these cases, a nontrivial rinse step is required to remove unbound target or a second hybridization step is required in the assay.

Nearly all assays for DNA sequences use the polymerase chain reaction (“PCR”) to amplify specific sequence segments from as little as a single copy of DNA to easily detected quantities (Reed et al., Practical Skills in Biomolecular Sciences, Addison Wesley Longman Limited, Edinburgh Gate, Harlow, England (1998); Walker et al., Molecular Biology and Biotechnology, The Royal Society of Chemistry, Thomas Graham House, Cambridge, UK (2000)). The use of PCR not only addresses sensitivity issues, but also effectively purifies samples to ameliorate the effects of large quantities of DNA that may not be of interest for a given assay. These features presently make the use of PCR nearly indispensable for the analysis of genomic DNA in spite of the development of a wide variety of innovative sensing approaches such as surface plasmon resonance (“SPR”) (Thiel et al., Anal. Chem. 69:4948-4956 (1997); Jordan et al., Anal. Chem. 69:4939-4947 (1997); Nelson et al., Anal. Chem. 73:1-7 (2001); He et al., J. Am. Chem. Soc. 122:9071-9077 (2000)), fluorescent microarrays (Sueda et al., Bioconjugate Chem. 13:200-205 (2002); Paris et al., Nucleic Acids Res. 26:3789-3793 (1998); Lepecq et al., Mol. Biol. 27:87-106 (1967)), assays based on semiconductor or metal nanoparticles (Bruchez et al., Science 281:2013-2016 (1998); Gerion et al., J. Am. Chem. Soc. 124:7070-7074 (2002); Chan et al., Science 281:2016-2018 (1998); Elghanian et al., Science 277:1078-1081 (1997); Taton et al., Science 289:1757-1760 (2000); Park et al., Science 295:1503-1506 (2002); Cao et al., Science 297:1536-1540 (2002); Maxwell et al., J. Am. Chem. Soc. 124:9606-9612 (2002); Dubertret et al., Nat. Biotech. 19:365-370 (2001); Sato et al., J. Am. Chem. Soc. 125:8102-8103 (2003)), and water-soluble conjugated polymer based sensors (Gaylord et al., J. Am. Chem. Soc. 125:896-900 (2003)). These techniques have been demonstrated mostly on purified synthesized oligonucleotides, but it may be possible to adapt some of them to be compatible with PCR amplified samples. Once PCR amplification is utilized, however, the merit of an assay is primarily determined by its simplicity rather than its sensitivity since additional amplification is straightforward. Most of the above approaches, as noted, require expensive instrumentation or involve time-consuming synthesis to modify DNA, substrates, or nanoparticles. In addition, it is usually necessary to conduct hybridization in the presence of substrates that introduce steric hindrance, leading to slow and inefficient binding between probe and target. As a result, post-processing of PCR amplified samples can be expensive and time-consuming (Rolfs et al., PCR: Clinical Diagnostics and Research, Springer-Verlag, Berlin Heidelberg (1992)).

Complexes between DNA and negatively charged gold nanoparticles have been studied for many years (Mirkin et al., Nature 382:607-609 (1996); Alivisatos et al., Nature 382:609-611 (1996)), and many creative schemes have exploited gold nanoparticles covalently functionalized with DNA sequences to bind specific target DNA sequences, either for nano-assembly or for oligonucleotide sensing (Elghanian et al., Science 277:1078-1081 (1997); Taton et al., Science 289:1757-1760 (2000); Park et al., Science 295:1503-1506 (2002); Cao et al., Science 297:1536-1540 (2002); Maxwell et al., J. Am. Chem. Soc. 124:9606-9612 (2002); Dubertret et al., Nat. Biotech. 19:365-370 (2001); Sato et al., J. Am. Chem. Soc. 125:8102-8103 (2003); Mirkin et al., Nature 382:607-609 (1996); Alivisatos et al., Nature 382:609-611 (1996); Chakrabarti et al., J. Am. Chem. Soc. 125:12531-12540 (2003); Loweth et al., Angew. Chem. Int. Ed. 38:1808-1812 (1999); Mbindyo et al., Adv. Mater. 13:249-254 (2001)).

Based on the foregoing, it would be desirable to provide an assay that utilizes conductive metal nanoparticles and target nucleic acid molecules that require no modification for detection of the target nucleic acid. Moreover, it would be desirable to provide an assay where hybridization is completely separate from detection so that it can be performed under optimal conditions without steric constraints of surface bound probes that slow hybridization dramatically and make it less efficient.

The present invention is directed to achieving these objectives and overcoming these and other deficiencies in the art.

### SUMMARY OF THE INVENTION

A first aspect of the present invention relates to a method for detecting presence or absence of a target nucleic acid molecule in a test solution (e.g., sample). This method includes the steps of: combining at least one single-stranded oligonucleotide probe with a test solution potentially including a target nucleic acid to form a hybridization solution, wherein the at least one single-stranded oligonucleotide probe and the test solution are combined under conditions effective to allow formation of a hybridization complex between the at least one single-stranded oligonucleotide probe and any target nucleic acid present in the test solution; exposing the hybridization solution to a plurality of metal nanoparticles under conditions effective to allow the at least one single-stranded oligonucleotide probe that remains unhybridized after said combining to associate electrostatically with the plurality of metal nanoparticles; and determining whether the at least one single-stranded oligonucleotide probe has hybridized to target nucleic acid or electrostatically associated with one or more of the plurality of metal nanoparticles, wherein hybridization to the target nucleic acid or electrostatic association with one or more metal nanoparticles is indicated by an optical property of the hybridization solution.

There are several embodiments for this aspect of the invention that are particularly preferred. One embodiment, designated a colorimetric assay, utilizes an unlabeled oligonucleotide probe and involves making the determination by detecting a color change of the hybridization solution after the step of exposing, whereby a color change indicates substantial aggregation of the plurality of metal nanoparticles in the presence of the target nucleic acid. If no color change (or an insignificant change) occurs, absence of the target nucleic acid is indicated. Another embodiment utilizes a fluorescently labeled oligonucleotide probe and involves determining whether or not fluorescence can be detected following exposure to the plurality of metal nanoparticles, whereby elimination of fluorescence indicates absence of a target nucleic acid and remaining fluorescence indicates its presence. If fluorescence by the labeled oligonucleotide probes remains, the oligonucleotide probes have formed duplexes and remain dissociated from the metal nanoparticles (i.e., no fluorescence quenching has occurred).

A second aspect of the present invention relates to a method for detecting a single nucleotide polymorphism (“SNP”) in a target nucleic acid molecule. This method is carried out by combining (i) a test solution including a target nucleic acid molecule and (ii) at least one first single-stranded oligonucleotide probe that has a nucleotide sequence that hybridizes to a region of the target nucleic acid molecule that may contain a single-nucleotide polymorphism, to form a test hybridization solution, wherein said combining is carried out under conditions effective to allow hybridization between the target nucleic acid molecule and the at least one first single-stranded oligonucleotide probe to form at least one hybridization complex; combining (i) a control solution including the target nucleic acid molecule and (ii) at least one second single-stranded oligonucleotide probe that has a nucleotide sequence that hybridizes perfectly to a region of the target nucleic acid molecule that does not contain a single-nucleotide polymorphism, to form a control hybridization solution, wherein said combining is carried out under conditions effective to allow hybridization between the target nucleic acid molecule and the at least one second single-stranded oligonucleotide probe to form at least one hybridization complex; exposing the test and control hybridization solutions, while maintaining the hybridization solutions at a temperature that is between the melting temperature of the at least one first single-stranded oligonucleotide probe and the melting temperature of the at least one second single-stranded oligonucleotide probe, to a plurality of metal nanoparticles under conditions effective to allow unhybridized probes in the hybridization solutions to electrostatically associate with the metal nanoparticles; and determining whether an optical property of the test and control hybridization solutions are substantially different, indicating the presence of the single nucleotide polymorphism in the target nucleic acid molecule.

A third aspect of the present invention relates to a method for detecting a SNP in a target nucleic acid molecule. This method is carried out by: combining (i) a solution including a target nucleic acid molecule and (ii) at least one first single-stranded oligonucleotide probe having a nucleotide sequence and a fluorescent label attached thereto, wherein the nucleotide sequence hybridizes to a region of the target nucleic acid molecule that may contain a single-nucleotide polymorphism, to form a hybridization solution, wherein said combining is carried out under conditions effective to allow hybridization between the target nucleic acid molecule and the at least one first single-stranded oligonucleotide probe to form at least one hybridization complex; exposing the hybridization solution to a plurality of metal nanoparticles under conditions effective to allow unhybridized probes in the hybridization solution to electrostatically associate with the metal nanoparticles; determining a temperature of the hybridization solution where quenching of the photoluminescence by the fluorescent label begins, said temperature representing the melting temperature; and comparing the melting temperature for the hybridization solution with a known melting temperature of a perfectly complementary probe, wherein a difference between the melting temperatures indicates the presence of the single nucleotide polymorphism in the target nucleic acid molecule.

A fourth aspect of the present invention relates to a method for detecting a target nucleic acid in a test solution. This method includes the steps of: subjecting a portion of a test solution potentially including a target nucleic acid to polymerase chain reaction and obtaining a product solution that includes single-stranded nucleic acid products of the polymerase chain reaction; combining at least one single-stranded oligonucleotide probe with the product solution to form a hybridization solution under conditions effective to allow formation of a hybridization complex between the at least one single-stranded oligonucleotide probe and any target nucleic acid present in the product solution; exposing the hybridization solution to a plurality of metal nanoparticles under conditions effective to allow any single-stranded nucleic acids in the hybridization solution to associate with the plurality of metal nanoparticles; and determining whether the at least one single-stranded oligonucleotide probe has hybridized to target nucleic acid or electrostatically associated with one or more of the plurality of metal nanoparticles, wherein hybridization to the target nucleic acid or electrostatic association with one or more metal nanoparticles is indicated by an optical property of the hybridization solution.

A fifth aspect of the present invention relates to a method of detecting a pathogen in a sample that includes the steps of obtaining a sample that may contain nucleic acid of a pathogen, and then performing a method of the present invention using an oligonucleotide probe specific for a target nucleic acid of the pathogen, wherein the step of determining that the at least one single-stranded oligonucleotide probe has hybridized to the target nucleic acid indicates presence of the pathogen.

A sixth aspect of the present invention relates to a method of genetic screening. This method is carried out by obtaining a sample, isolating DNA from the sample, amplifying the DNA isolated from the sample, and then performing a method of the present invention using an oligonucleotide probe specific for diagnosing a genetic condition, hereditary condition, or the like, wherein the step of determining that the at least one single-stranded oligonucleotide probe has hybridized to the target nucleic acid indicates predisposition to the genetic condition, hereditary condition, or identification of an organism.

A seventh aspect of the present invention relates to a method of detecting a protein in a sample. This method is carried out by obtaining a sample, performing an immuno-polymerase chain reaction procedure using the sample, wherein the immuno-polymerase chain reaction procedure results in amplification of a nucleic acid that is conjugated to a protein, and then performing a method of the present invention using an oligonucleotide probe specific for the nucleic acid that is conjugated to the protein (or its complement), wherein the step of determining that the at least one single-stranded oligonucleotide probe has hybridized to the target nucleic acid indicates that the protein is present in the sample.

An eighth aspect of the present invention relates to a method of quantifying the amount of amplified nucleic acid prepared by polymerase chain reaction. This method is carried out by providing two or more fluorescently labeled oligonucleotide primers that each have a nucleotide sequence capable of hybridizing to a nucleic acid molecule, or its complement, to be amplified; performing polymerase chain reaction using a target nucleic acid molecule and/or its complement, and the provided fluorescently labeled oligonucleotide primers; and performing the fluorimetric method of the present invention on a sample obtained after said performing polymerase chain reaction, wherein the level of fluorescence detected from the sample indicates the amount of primer that has been incorporated into an amplified nucleic acid molecule.

A ninth aspect of the present invention relates to kits containing various components that will allow a user to perform one or more methods of the present invention. According to one embodiment, the kits minimally include a first container that contains a colloidal solution including metal nanoparticles and a second container that contains an aqueous solution including at least one single-stranded oligonucleotide probe having a nucleotide sequence that is substantially complementary to a target nucleic acid molecule.

Assays and kits of the present invention involve the use of metal nanoparticles and nucleic acid molecules, harnessing the electrostatic interactions between the metal nanoparticles and nucleic acid molecules. In particular, applicants have identified four unique interactions that can be harnessed by the assays and materials of the present invention. These include: (1) the discovery that under certain conditions single stranded nucleic acid will adsorb on negatively charged metal nanoparticles while double stranded nucleic acid molecules will not; (2) adsorption of single stranded nucleic acid molecules onto the metal nanoparticles suspended in a colloidal solution stabilizes the nanoparticles against salt-induced aggregation; (3) the adsorption rate for single stranded nucleic acid molecules depends on the sequence length; and (4) the adsorption rate for single stranded nucleic acid molecules depends on the temperature of the solution.

The essential difference between the electrostatic properties of single-stranded and double-stranded DNA probably arises because ss-DNA can uncoil sufficiently to expose its bases while ds-DNA has a stable double helix geometry that always presents the negatively charged phosphate backbone (Watson, The Double Helix: A Personal Account of the Discovery of the Structure of DNA, Weidenfeld and Nicholson, London (1968); Bloomfield et al., Nuclei Acids: Structures, Properties, and Functions, University Science Books, Sausalito, Calif. (1999), each of which is hereby incorporated by reference in its entirety). Conductive metal nanoparticles in solution are typically stabilized by adsorbed negative ions (e.g., citrate) whose repulsion prevents the strong Van der Waals attraction between the metal particles from causing them to aggregate (Hunter, Foundations of Colloid Science, Oxford University Press Inc., New York (2001); Shaw, Colloid and Surface Chemistry, Butterworth-Heinemann Ltd., Oxford (1991), each of which is hereby incorporated by reference in its entirety). Repulsion between the charged phosphate backbone of ds-DNA and the adsorbed citrate ions dominates the electrostatic interaction between the metal nanoparticle and ds-DNA so that ds-DNA will not adsorb. Because the ss-DNA is sufficiently flexible to partially uncoil its bases, they can be exposed to the metal nanoparticles. Under these conditions, the negative charge on the backbone is sufficiently distant so that attractive Van der Waals forces between the bases and the metal nanoparticle are sufficient to cause ss-DNA to stick to the metal. The same mechanism is not operative with ds-DNA because the duplex structure does not permit the uncoiling needed to expose the bases. In the present invention, the selective adsorption of ss-DNA metal nanoparticles is documented. In addition, it is shown that adsorption of ss-DNA stabilizes the metal nanoparticles against aggregation at concentrations of salt that would ordinarily screen the repulsive interactions of the citrate ions. The color of metal nanoparticles is determined principally by surface plasmon resonance and this is dramatically affected by aggregation of the nanoparticles (Link et al., Intl. Reviews in Physical Chemistry 19:409-453 (2000); Kreibig et al., Surface Science 156:678-700 (1985); Quinten et al., Surface Science 172:557-577 (1986), each of which is hereby incorporated by reference in its entirety). The difference in the electrostatic properties of ss-DNA and ds-DNA can be used to design a simple colorimetric hybridization assay. The assay can be used for sequence specific detection of untagged oligonucleotides using unmodified commercially available materials. The assay is easy to implement for visual detection at the level of 100 femtomoles, and it is shown that it is easily adapted to detect single base mismatches between probe and target. Also presented herein are initial studies of how length mismatches between target and probe sequence affect the propensity for oligonucleotides to adsorb on metal nanoparticles.

By harnessing the above-identified interactions in the assays and kits of the present invention, the present invention affords methods of detecting target nucleic acids that offer a number of benefits over previously developed detection procedures. Some of these benefits include: no target labeling is required; the assays occur in solution, allowing for detection of the target nucleic acid in less than about 10 minutes (which is significantly faster than chip or surface-based assays that tend to slow down the hybridization process); the detection procedure is temporally separated from the hybridization procedure so that the hybridization process can be optimized with little or no regard to the detection procedure; and the assays can be performed using commercially available materials. The two basic embodiments of the present invention, a colorimetric assay and a fluorimetric assay, afford significant benefits. The colorimetric assay can be performed without the need for expensive detection instrumentation, such as fluorescence microscopes or photomultipliers. Detection of a positive or negative result in the colorimetric assay can be assessed by naked eye of an observer. The assays are extremely sensitive, capable of detecting target nucleic acids at concentrations in the femtomolar range (or less in the case of the fluorescent approach), capable of discriminating between complex mixtures of nucleic acid, and capable of discriminating between wild-type targets and those bearing SNPs. Detection of SNPs in genomic DNA is particularly challenging, but is at the forefront of diagnostic technology since it has been associated with a number of hereditary conditions and cancers and is likely to be responsible for many more (Friedberg, Nature 421:436-439 (2003); Futreal et al., Nature 409:850-852 (2002), each of which is hereby incorporated by reference in its entirety).

### BRIEF DESCRIPTION OF THE DRAWINGS

FIG. 1 is a pictorial representation of the colorimetric method for differentiating between single and double stranded oligonucleotides; and consequently selective oligonucleotide detection. The circles represent colloidal metal (e.g. gold) nanoparticles.

FIG. 2 is a pictorial representation of the fluorimetric method for selective oligonucleotide detection. The red stars in panels A, B, and D represent identifiable (i.e., unquenched) fluorescence from the fluorescence label on the probe strands. The thin green strands and the thick green strands represent single-strand and double-strand nucleic acid molecules, respectively. The circles in panels C and D represent metal (e.g., gold) nanoparticles. Hybridization between the oligonucleotide probes and target nucleic acid molecules occur before introducing metal nanoparticles. When the nanoparticles are introduced into the hybridization solution where DNA duplex formation did not occur, the fluorescence from the tag on the probe is quenched (panel C). When the nanoparticles are introduced into solution where hybridization occurred, fluorescence from the tag on duplex-forming probes is observed (panel D).

FIG. 3 is a schematic protocol of protein detection combining immuno-PCR with the methods of the present invention.

FIGS. 4A-B provide evidence for preferential adsorption of ss-DNA on gold nanoparticles. FIG. 4A is a graphical illustration of fluorescence emitted from rhodamine red attached to ss-DNA (dashed) and ds-DNA (solid). The fluorescence spectra were recorded from mixtures consisting of the trial hybridization solution (final concentration of the dye labeled ss-DNA: 50 nM), 500 μL of gold colloid, and 500 μL of 10 mM phosphate buffer solution (PBS) containing 0.1 M NaCl. The ss-DNA (dashed) curve was recorded from the mixture containing the probe and its non-complementary target (nc-target). Dot curve was recorded from the mixture containing the probe and its complementary target (c-target). FIG. 4B is a graphical illustration of Surface Enhanced Resonant Raman Scattering (“SERRS”) from Rhodamine Green tagged on ss-DNA (solid) and ds-DNA (dashed). SERRS was recorded from the mixture of 5 picomole probe and 5 picomole nc-target (solid curve) or 5 picomole c-target (dashed curve), and 100 μL of 10 mM PBS containing 0.5 M NaCl, as well as 300 μL silver colloid. The Raman modes at 1645, 1558, 1509, and 1363 cm⁻¹ are aromatic C—C stretching modes of the core of rhodamine green, while the Raman modes at 1279 and 1182 cm⁻¹ are rhodamine C—O—C stretching and C—C stretching vibrations, respectively.

FIGS. 5A-C show colorimetric detection of oligonucleotide hybridization. FIG. 5A is a graph showing absorption spectra of gold colloid (diamonds) and the mixtures containing ss-DNA1 (circles), ss-DNA2 (triangles), and ds-DNA from the hybridization of ss-DNA1 and ss-DNA2 (squares), respectively. The gold colloid was diluted with water to the same concentration as in the mixtures. The mixtures contained trial hybridization solution (5 μL (60 μM) ss-DNA in salt buffer solution) added to 500 μL of 17 nM gold colloid, followed by 200 μL of 10 mM PBS and 0.2 M NaCl). FIG. 5B is a graphical illustration of the ratio of the absorbance at 520 nm to the absorbance at 700 nm versus oligonucleotide concentration expressed in number of DNA per gold nanoparticle. The DNA sequences and the mixture are the same as in FIG. 5A, except for variation of the amount of DNA. FIG. 5B is a photograph showing colorimetric detection of a DNA sequence fragment characteristic of Severe Acute Respiratory Syndrome (“SARS”) virus (Drosten et al., The New England Journal of Medicine 348:1967-1976 (2003), which is hereby incorporated by reference in its entirety). All solutions contained 120 picomoles of probe, 200 μL gold colloid, and 100 μL of 10 mM PBS and 0.2 M NaCl. The ratio of the amount of target to the amount of probe in the solutions was 0, 0.2, 0.4, 0.6, and 1 (from left to right), respectively.

FIGS. 6A-E show colorimetric detection of targets in mixtures, low concentrations, low amounts, and with single base mismatches. FIG. 6A is a photograph showing detection of a target sequence in a mixture. 3.5 μL of trial hybridization solution was mixed with 300 μL of gold colloid and 300 μL of 10 mM phosphate buffer solution containing 0.2 M NaCl. The complementary target contained in the solutions from left to right were 50%, 40%, 30%, and 0% of the total oligonucleotide concentration with non-complementary target making up the remainder. All solutions contained the 105 picomoles of probe, equal to the total of complementary target and non-complementary target. FIG. 6B is a photograph showing detection of target DNA in low concentration solution. 100 μL of gold colloid was diluted in 300 μL water, mixed with 1 μL trial hybridization solution and 300 μL of 10 mM phosphate buffer solution containing 0.3 M NaCl (final target concentration: 4.3 nM). The vial on the left contained unmatched ss-DNA strands while the vial on the right contained complementary strands. FIG. 6C is a photograph showing detection of small amounts of target. 5 μL of gold colloid was mixed with 0.2 μL of trial hybridization solutions containing 0.3 μM oligonucleotide then mixed with 3 μL of 10 mM phosphate buffer solution containing 0.2 M NaCl. The resulting droplets of non-complementary ss-DNA mixture (left) and complementary ss-DNA (right) each containing 60 femtomoles were placed on inverted plastic vials for viewing. FIG. 6D is a photograph showing identification of single base pair mismatch in ds-DNA via dehybridization kinetics in water. 1 μL of ds-DNA solution dehybridized in 100 μL water for 0, 1, and 2 minutes respectively, then mixed with 300 μl of gold nanoparticles and 300 μL of 10 mM phosphate buffer solution 0.3 M NaCl (final ds-DNA concentration: 0.043 μM). The solution in the left vial of each dehybridization time group contained ds-DNA with a single base pair mismatch while the right vial contained perfectly matched target and probe strands. The red color indicates that part of ds-DNA has dehybridized. FIG. 6E is a photograph showing identification of single base pair mismatch in ds-DNA via dehybridization kinetics in gold colloid. 1 μL oligonucleotide and 300 μL of gold nanoparticles were ultrasonicated for 0.5, 1, and 2 minutes, respectively, and then mixed with 300 μL of 10 mM phosphate buffer solution 0.3 M NaCl (final target concentration: 0.05 μM). The solution in the left vial of each dehybridization time group contained ds-DNA with a single base pair mismatch while the right vial contained perfectly matched target and probe strands. The red color indicates that part of ds-DNA has dehybridized. The oligonucleotide sequences are identified in the text.

FIGS. 7A-B show that gold nanoparticles preferentially quench the fluorescence from fluorophores labeled on ss-DNA. FIG. 7A is a graph showing the fluorescence spectra of the mixtures of 5 μL (10 μM) trial hybridized solution of rhodamine red labeled ss-DNA probe and its complementary target (solid squares), or non-complementary target (open squares), 500 μL of gold colloid and 500 μL of 10 mM PBS containing 0.1 M NaCl. FIG. 7B is a graph showing the fluorescence image intensity profile measured with a confocal fluorescence microscope. 0.5 μL (0.1 μM) of the trial hybridization solution was mixed with 500 μL of the diluted gold colloid (diluted with deionized water by factor 20) and 500 μL of 10 mM PBS containing 0.1 M NaCl. Solid circles were recorded from 2 μL of the mixture containing complementary target; open circles from 2 μL of the mixture containing non-complementary target.

FIGS. 8A-B show detection of long target and long target in a mixture. FIG. 8A is a graph showing the method working with long target. The fluorescence spectra were recorded from the solutions containing complementary target a (solid squares), complementary target b (open squares), and non-complementary target c (solid triangles), respectively. The solution contained 4 μL (10 μM) of trial hybridized solution, 500 μL gold colloid, and 500 μL of 10 mM PBS containing 0.1 M NaCl. FIG. 8B is a graph showing the method working with long target in a mixture. The fluorescence spectra were recorded from mixtures containing 1% complementary target a (solid squares), 1% complementary target b (open squares), and non-complementary target (solid triangles), respectively. The components of oligonucleotides in the trial hybridized solution contained 10 picomolar non-complementary target, 0.5 picomolar probe, and 0.1 picomolar candidates. The mixtures were made up of 0.5 μL of trial hybridized solutions, 500 μL gold colloid (diluted with 250 μL water), and 500 μL of 10 mM PBS containing 0.1M NaCl.

FIGS. 9A-B show single base-pair mismatch detection. FIG. 9A is a graph showing the probe binding in the middle of long target a and target a′. FIG. 9B is a graph showing the probe binding at one end of long target b and complementary target b′. The fluorescence spectra for single base-pair mismatch detection were recorded from mixtures containing 1 μL (10 μM) trial hybridized solution (same amount of the probe and the target) warmed in 46° C. water bath, 500 μL gold colloid, and 500 μL of 10 mM PBS and 0.1 M NaCl. Solid squares were recorded from the mixtures containing perfect matched ds-DNA and open squares from the mixtures containing ds-DNA with one base-pair mismatch.

FIGS. 10A-B show simultaneous multiple target detection. FIG. 10A is a graph showing excitation at 570 nm, which is absorption maximum of rhodamine red tagged on probe 1. FIG. 10B is a graph showing excitation at 648 nM, which is absorption maximum of cy5 tagged on probe 2. (Note: The second peak of the spectrum (solid squares) in FIG. 10B is the emission of cy5 tagged on probe 2 excited by 570 nm.)

FIGS. 11A-D show adsorption of ss-DNA to gold nanoparticles. FIG. 11A graphically illustrates absorption spectra of 300 μL gold colloid and 100 μL deionized water (red), 100 μL of 10 mM PBS (0.2 M NaCl) (blue), 300 picomoles 24 base ss-DNA first, then 100 μL of 10 mM PBS (0.2 M NaCl) (green). FIG. 11B is a graph showing photoluminescence intensity versus time following addition of 4 picomoles rhodamine red tagged ss-DNAs to 1000 μL gold colloid. 10 mer (red), 24 mer (green) and 50 mer (blue). FIG. 11C graphically illustrates absorption spectra of the mixture of 200 picomoles ss-DNA (50 mer) and 300 μL gold nanoparticles heated at different temperature for two minutes, followed by addition of 300 μL of 10 mM PBS (0.2 M NaCl). 22° C. (blue), 45° C. (cyan), 70° C. (green), and 95° C. (red). FIG. 11D graphically illustrates the fluorescence spectra of the hybridized solutions of rhodamine red labeled 15 mer ss-DNA, 50 mer ss-DNA, and gold colloid, the 15 mer binding to 50 mer at middle (red), at end (green) and nowhere (blue). The lower inset schematically illustrates the binding positions between 15 mer and 50 mer. The upper inset contains color photographs of the corresponding mixtures (from left to right) with no fluorescent label on the 15 mer.

FIG. 12 is a schematic of the interaction between negatively charged metal nanoparticules and ss-DNA. The wedge-like structure (left) represents the metal nanoparticle, and the structure (right) represents a ss-nucleic acid having a phosphate backbone (solid vertical line) and nucleotide bases (horizontal lines).

FIG. 13 shows identification of PCR amplified DNA sequences. FIG. 13A is a schematic of the detection protocol. The mixture of PCR product and probes is denatured and annealed below the melting temperature of the complementary probes, followed by addition of gold colloid. The long blue and green lines represent the PCR amplified DNA fragments and the pink and light blue medium bars the excess PCR primers. The short blue and green bars are complementary probes that bind, resulting in gold nanoparticle aggregation (purple color). The short purple and orange bars are non-complementary probes that do not bind and adsorb to the gold nanoparticles, preventing nanoparticle aggregation and leaving the solution pink. FIG. 13B is a color photograph of the resulting solutions with complementary probes (a) and non-complementary probes (b). 8 μL PCR product, 3.5 picomoles probe and 70 μL gold colloid were used in each vial.

FIGS. 14A-B show single base-pair mismatch detection. FIG. 14A illustrates the detection strategy. The red spots on long green and blue lines represent positions of a potential SNP. The long green and blue lines are the complementary sequences of PCR amplified DNA fragment. The short green and blue bars are probes complementary to parts of the wild type sequence of PCR amplified DNA fragment as illustrated. FIG. 14B is a photograph showing detection of a single base-pair mismatch. Vials b, d, and f contain PCR product with probes overlapping the single-base mismatch while vials a, c, and e contain PCR product with probes not overlapping the single base pair mismatch. Photographs were taken of the mixtures annealed at 50° C. (a, b), 54° C. (c, d) and 58° C. (e, f). 8 μL PCR product, 3.5 picomoles probe and 70 μL gold colloid were used in each vial.

FIGS. 15A-B illustrate single base-pair mismatch detection using RNA probes and RNA targets. The symbols shown in FIGS. 15A-B are as follows: ds: duplex; ds′: duplex containing mismatch; ss: control.

### DETAILED DESCRIPTION OF THE INVENTION

The methods of the present invention can be used to detect the presence (or substantial absence) of a target nucleic acid molecule in a sample or test solution. Basically, the method involves combining at least one single-stranded oligonucleotide probe and the test solution under conditions effective to allow formation of a hybridization complex between the at least one single-stranded oligonucleotide probe and any target nucleic acid present in the test solution. If no target nucleic acid or substantially no target nucleic acid is present, then no hybridization complex or substantially no hybridization complex will form. After allowing for hybridization to occur (i.e., if hybridization between the probe and target is possible), the hybridization solution is exposed to a plurality of metal nanoparticles under conditions effective to allow any unhybridized probe to associate electrostatically with the plurality of metal nanoparticles. A determination is then made whether the at least one single-stranded oligonucleotide probe has hybridized to target nucleic acid or electrostatically associated with one or more of the plurality of metal nanoparticles. This determination is made according to an optical property of the hybridization solution, as discussed below.

The target nucleic acid molecule that is intended to be detected can be DNA or RNA. The DNA or RNA can be isolated directly from samples (i.e., concentrated to be free of cellular debris) and then tested, if present in sufficient quantities, or it can first be amplified by polymerase chain reaction (“PCR”) or reverse-transcription PCR. Thus, the DNA to be detected can be amplified cDNA. Because the DNA can be amplified cDNA, the cDNA can also have incorporated therein synthetic, natural, or structurally modified nucleoside bases.

The target nucleic acid molecule can also be from any source organism (e.g., human or another animal, virus, bacteria, insect, plant, etc.).

As an alternative, the target nucleic acid can contain a nucleotide sequence coupled or otherwise conjugated to a protein or polypeptide. In such case, detection of the target nucleic acid directly confirms presence of the protein or polypeptide. Alternatively, the target nucleic acid can contain a nucleotide sequence coupled or otherwise conjugated to a protein or polypeptide that participates in an immuno-PCR procedure; the subsequently amplified target cDNA confirms indirectly the presence of the target nucleic acid in a sample to be tested (i.e., absence of the target cDNA confirms that the target is not present in the initial sample).

The single-stranded oligonucleotide probes that can be used in the present invention can either be unlabeled or they can be conjugated or otherwise coupled to a fluorescent label. Coupling of the fluorescent label to the oligonucleotide probe can be achieved using known nucleic acid-binding chemistry or by physical means, such as through ionic, covalent or other forces well known in the art (see, e.g., Dattagupta et al., Analytical Biochemistry 177:85-89 (1989); Saiki et al., Proc. Natl. Acad. Sci. USA 86:6230-6234 (1989); Gravitt et al., J. Clin. Micro. 36:3020-3027 (1998), each of which is hereby incorporated by reference in its entirety). Either a terminal base or another base near the terminal base can be bound to the fluorescent label. For example, a terminal nucleotide base of the oligonucleotide probe can be modified to contain a reactive group, such as (without limitation) carboxyl, amino, hydroxyl, thiol, or the like.

The fluorescent label can be any fluorophore that can be conjugated to a nucleic acid and preferably has a photoluminescent property that can be detected and easily identified with appropriate detection equipment. Exemplary fluorescent labels include, without limitation, fluorescent dyes, semiconductor quantum dots, lanthanide atom-containing complexes, and fluorescent proteins. The fluorophore used in the present invention is characterized by a fluorescent emission maxima that is detectable either visually or using optical detectors of the type known in the art. Fluorophores having fluorescent emission maxima in the visible spectrum are preferred.

Exemplary dyes include, without limitation, Cy2™, YO-PRO™-1, YOYO™-1, Calcein, FITC, FluorX™, Alexa™, Rhodamine 110, 5-FAM, Oregon Green™ 500, Oregon Green™ 488, RiboGreen™, Rhodamine Green™, Rhodamine 123, Magnesium Green™, Calcium Green™, TO-PRO™-1, TOTO®-1, JOE, BODIPY® 530/550, Dil, BODIPY® TMR, BODIPY® 558/568, BODIPY® 564/570, Cy3™, Alexa™ 546, TRITC, Magnesium Orange™, Phycoerythrin R&B, Rhodamine Phalloidin, Calcium Orange™, Pyronin Y, Rhodamine B, TAMRA, Rhodamine Red™, Cy3.5™, ROX, Calcium Crimson™, Alexa™ 594, Texas Red®, Nile Red, YO-PRO™-3, YOYO™-3, R-phycocyanin, C-Phycocyanin, TO-PRO™-3, TOTO®-3, DiD DilC(5), Cy5™, Thiadicarbocyanine, and Cy5.5™. Other dyes now known or hereafter developed can similarly be used as long as their excitation and emission characteristics are compatible with a light source and non-interfering with other fluorophores that may be present (i.e., not capable of participating in fluorescence resonant energy transfer or FRET).

Attachment of dyes to the oligonucleotide probe can be carried out using any of a variety of known techniques allowing, for example, either a terminal base or another base near the terminal base to be bound to the dye. For example, 3′-tetramethylrhodamine (TAMRA) may be attached using commercially available reagents, such as 3′-TAMRA-CPG, according to manufacturer's instructions (Glen Research, Sterling, Va.). Other exemplary procedures are described in, e.g., Dubertret et al., Nature Biotech. 19:365-370 (2001); Wang et al., J. Am. Chem. Soc., 125:3214-3215 (2003); Bioconjugate Techniques, Hermanson, ed. (Academic Press) (1996), each of which is hereby incorporated by reference in its entirety.

Exemplary proteins include, without limitation, both naturally occurring and modified (i.e., mutant) green fluorescent proteins (Prasher et al., Gene 111:229-233 (1992); PCT Application WO 95/07463, each of which is hereby incorporated by reference in its entirety) from various sources such as Aequorea and Renilla; both naturally occurring and modified blue fluorescent proteins (Karatani et al., Photochem. Photobiol. 55(2):293-299 (1992); Lee et al., Methods Enzymol. (Biolumin. Chemilumin.) 57:226-234 (1978); Gast et al., Biochem. Biophys. Res. Commun. 80(1):14-21 (1978), each of which is hereby incorporated by reference in its entirety) from various sources such as Vibrio and Photobacterium; and phycobiliproteins of the type derived from cyanobacteria and eukaryotic algae (Apt et al., J. Mol. Biol. 238:79-96 (1995); Glazer, Ann. Rev. Microbiol. 36:173-198 (1982); Fairchild et al., J. Biol. Chem. 269:8686-8694 (1994); Pilot et al., Proc. Natl. Acad. Sci. USA 81:6983-6987 (1984); Lui et al., Plant Physiol. 103:293-294 (1993); Houmard et al., J. Bacteriol. 170:5512-5521 (1988), each of which is hereby incorporated by reference in its entirety), several of which are commercially available from ProZyme, Inc. (San Leandro, Calif.). Other fluorescent proteins now known or hereafter developed can similarly be used as long as their excitation and emission characteristics are compatible with the light source and non-interfering with other fluorophores that may be present.

Attachment of fluorescent proteins to the oligonucleotide probe can be carried out using substantially the same procedures used for tethering dyes to the nucleic acids, see, e.g., Bioconjugate Techniques, Hermanson, ed. (Academic Press) (1996), which is hereby incorporated by reference in its entirety.

Nanocrystal particles or semiconductor nanocrystals (also known as Quantum Dot™ particles), whose radii are smaller than the bulk exciton Bohr radius, constitute a class of materials intermediate between molecular and bulk forms of matter. Quantum confinement of both the electron and hole in all three dimensions leads to an increase in the effective band gap of the material with decreasing crystallite size. Consequently, both the optical absorption and emission of semiconductor nanocrystals shift to the blue (higher energies) as the size of the nanocrystals gets smaller. When capped nanocrystal particles of the invention are illuminated with a primary light source, a secondary emission of light occurs at a frequency that corresponds to the band gap of the semiconductor material used in the nanocrystal particles. The band gap is a function of the size of the nanocrystal particle. As a result of the narrow size distribution of the capped nanocrystal particles, the illuminated nanocrystal particles emit light of a narrow spectral range resulting in high purity light. Particles size can be between about 1 nm and about 1000 nm in diameter, preferably between about 2 nm and about 50 nm, more preferably about 5 nm to about 20 nm.

Fluorescent emissions of the resulting nanocrystal particles can be controlled based on the selection of materials and controlling the size distribution of the particles. For example, ZnSe and ZnS particles exhibit fluorescent emission in the blue or ultraviolet range (˜400 nm or less); Au, Ag, CdSe, CdS, and CdTe exhibit fluorescent emission in the visible spectrum (between about 440 and about 700 nm); InAs and GaAs exhibit fluorescent emission in the near infrared range (˜1000 nm), and PbS, PbSe, and PbTe exhibit fluorescent emission in the near infrared range (i.e., between about 700-2500 nm). By controlling growth of the nanocrystal particles it is possible to produce particles that will fluoresce at desired wavelengths. As noted above, smaller particles will afford a shift to the blue (higher energies) as compared to larger particles of the same material(s).

Preparation of the nanocrystal particles can be carried out according to known procedures, e.g., Murray et al., MRS Bulletin 26(12):985-991 (2001); Murray et al., IBM J. Res. Dev. 45(1):47-56 (2001); Sun et al., J. Appl. Phys. 85(8, Pt. 2A): 4325-4330 (1999); Peng et al., J. Am. Chem. Soc. 124(13):3343-3353 (2002); Peng et al., J. Am. Chem. Soc. 124(9):2049-2055 (2002); Qu et al., Nano Lett. 1(6):333-337 (2001); Peng et al., Nature 404(6773):59-61 (2000); Talapin et al., J. Am. Chem. Soc. 124(20):5782-5790 (2002); Shevenko et al., Advanced Materials 14(4):287-290 (2002); Talapin et al., Colloids and Surfaces, A: Physiochemical and Engineering Aspects 202(2-3):145-154 (2002); Talapin et al., Nano Lett. 1(4):207-211 (2001), each of which is hereby incorporated by reference in its entirety. Alternatively, nanocrystal particles can be purchased from commercial sources, such as Evident Technologies.

Attachment of a nanocrystal particle to the oligonucleotide probe can be carried out using substantially the same procedures used for tethering dyes thereto. Details on these procedures are described in, e.g., Bioconjugate Techniques, Hermanson, ed. (Academic Press) (1996), which is hereby incorporated by reference in its entirety.

Exemplary lanthanide atoms include, without limitation, Ce, Pr, Nd, Pm, Sm, Eu, Gd, Th, Dy, Ho, Er, Tm, Yb, and Lv. Of these, Nd, Er, and Th are preferred because they are commonly used in fluorescence applications. Attachment of a lanthanide atom (or a complex containing the lanthanide atom) to the oligonucleotide probe can be carried out using substantially the same procedures used for tethering dyes thereto. Details on these procedures are described in, e.g., Bioconjugate Techniques, Hermanson, ed. (Academic Press) (1996), which is hereby incorporated by reference in its entirety.

When multiple probes are used and each is conjugated to a fluorescent label, it is preferable that the fluorescent labels can be distinguished from one another using appropriate detection equipment. That is, the fluorescent emissions of one fluorescent label should not overlap or interfere with the fluorescent emissions of another fluorescent label being utilized. Likewise, the absorption spectra of any one fluorescent label should not overlap with the emission spectra of another fluorescent label (which may result in fluorescent resonance energy transfer that can mask emissions by the other label).

The single-stranded oligonucleotide probe can be formed of either RNA or DNA, and can contain one or more modified bases, one or more modified sugars, one or more modified backbones, or combinations thereof. The modified bases, sugars, or backbones can be used either to enhance the affinity of the probe to a target nucleic acid molecule or to allow for conjugation to a fluorescent label. Exemplary forms of modified bases are known in the art and include, without limitation, alkylated bases, alkynylated bases, thiouridine, and G-clamp (Flanagan et al., Proc. Natl. Acad. Sci. USA 30:3513-3518 (1999), which is hereby incorporated by reference in its entirety). Exemplary forms of modified sugars are known in the art and include, without limitation, LNA, 2′-O-methyl, 2′-O-methoxyethyl, and 2′-fluoro (see, e.g., Freier and Attmann, Nucl. Acids Res. 25:4429-4443 (1997), which is hereby incorporated by reference in its entirety). Exemplary forms of modified backbones are known in the art and include, without limitation, phosphoramidates, thiophosphoramidates, and alkylphosphonates. Other modified bases, sugars, and/or backbones can, of course, be utilized.

The single-stranded oligonucleotide probes can be of any length that is suitable to allow for rapid hybridization to target nucleic acids (if present) in the test solution, and rapid electrostatic association with metal nanoparticles later introduced into the test solution. By rapid, it is intended that the single-stranded oligonucleotide probe can electrostatically associate with metal nanoparticles at a rate that is greater (preferably by at least an order of magnitude) than the rate of association with other nucleic acids in the test solution prior to introduction of the oligonucleotide probe. By way of example and without limitation, the single-stranded oligonucleotide probes are preferably between about 10 and about 50 nucleotides in length, more preferably between about 10 and 30 nucleotides in length, most preferably between about 12 and 20 nucleotides in length.

The single-stranded oligonucleotide probes can have their entire length or any portion thereof targeted to hybridize to the target nucleic acid. It is preferable for the oligonucleotide probe to have a nucleotide sequence that is 100 percent or perfectly complementary to part of the target nucleic acid sequence.

The amount of oligonucleotide probe introduced into the test solution can be determined based upon the total amount of metal nanoparticles to be introduced into the hybridization solution and/or the total amount of target nucleic acid that is believed to be present.

For the colorimetric assay, it is preferable that the amount of oligonucleotide probe is at least slightly greater than the amount of metal nanoparticles present in the hybridization solution (i.e., greater than a 1:1 ratio), more preferably greater than about 10:1, and up to about 30:1. A reasonable match in the amounts of probe and target used are desirable for optimization of the assay. If the amount of nucleic acid is a sample can be reasonable estimated, then the ratio of probe:target should be between about 0.3:1 and about 3:1. If reasonable estimates cannot be made, then concentration series can be performed.

For the fluorescent assay, the relative concentrations of target and probe in the trial solution are not critical. Instead, an excess of metal nanoparticles is utilized so that all the unhybridized probes will be quenched (and excess target does not produce fluorescence).

When more than one single-stranded oligonucleotide probe is utilized at a time, the same criteria disclosed above can be taken into consideration.

The oligonucleotide probe can be synthesized using standard synthesis procedures or ordered from commercial vendors, such as Midland Certified Reagent Co. (Midland, Tex.) and Integrated DNA Technologies, Inc. (Coralville, Iowa).

For the colorimetric assay, the metal nanoparticles are preferably provided in the form of a solution that contains a colloidal suspension of the metal nanoparticles. For the fluorimetric assay, the colloidal metal nanoparticles can be provided either in solution or they can be immobilized on a solid surface (e.g., glass surface) using standard coupling protocols. By immobilizing the colloidal metal particles, there is no need to prepare or provide a stable colloidal metal nanoparticle solution. Commercially, this would be the more desirable approach.

The metal nanoparticles can be formed of any conductive metal or metal alloy that allows the nanoparticle to be capable of electrostatically associating with a single-stranded nucleic acid molecule or aggregating with other metal nanoparticles under appropriate conditions. (Prior to use in the present invention, it should be appreciated that the colloidal suspension maintains the metal nanoparticles in a stable environment in which they are substantially free of aggregation.) Importantly, the metal nanoparticles do not significantly associate electrostatically with hybridization complexes (that is, double-stranded nucleic acid molecules). Exemplary metal nanoparticles include, without limitation, gold nanoparticles, silver nanoparticles, platinum nanoparticles, mixed metal nanoparticles (e.g., gold shell surrounding a silver core), and combinations thereof.

Suspensions of colloidal metal nanoparticles can be formed using the procedures described in Grabar et al., Anal. Chem. 67:735-743 (1995), which is hereby incorporated by reference in its entirety. The metal nanoparticles preferably do not contain any ligands conjugated or otherwise bound to their outer surface. They are, however, stabilized by, e.g., citrate ions in the solution. The colloidal suspension preferably contains metal nanoparticles of between about 5 nm and about 500 nm, most preferably between about 10 nm and 30 nm.

In practicing the assay, the detection of hybridization between probe and target can be achieved in one of two preferred approaches: a colorimetric approach or a fluorimetric approach. Each has a distinct advantage over the other and can be employed as desired.

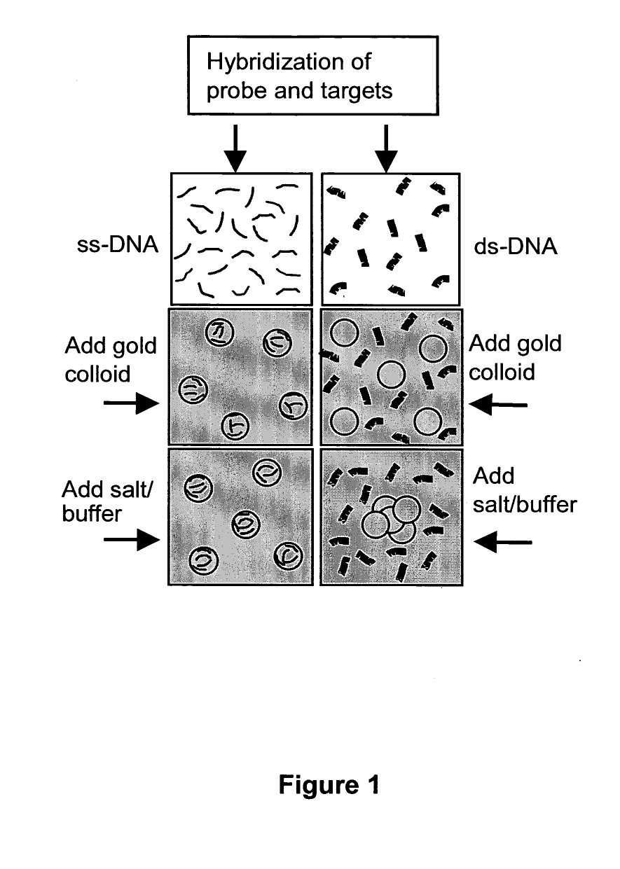

In a colorimetric assay (in which the probe can be unlabeled), the optical property of the hybridization solution is the visible color thereof. A color change of the hybridization solution can be brought about by inducing aggregation of the plurality of metal nanoparticles as illustrated in FIG. 1. The colorimetric assay is particularly useful when quantification is not necessary and where expensive detection equipment is unavailable. Detection of the color change in the hybridization solution can be carried out by naked eye observation of a user (i.e., the person performing the assay).

Aggregation will only occur if an insubstantial number of oligonucleotide probes has electrostatically associated with the metal nanoparticles. If a substantial number of oligonucleotide probes has electrostatically associated with the metal nanoparticles (on average greater than about one or two per nanoparticle), aggregation will be inhibited significantly. Aggregation (color change) indicates that the target nucleic acid was present in the test solution. Induction of aggregation can be carried out by introducing a salt solution into the hybridization solution, with the salt being of sufficient concentration to alter the electrostatic properties of the metal nanoparticles, thereby promoting their aggregation. The salt solution preferably comprises a Na⁺ concentration of between about 0.01 and about 1 M, more preferably between about 0.1 and about 0.3 M. The introduction of the salt solution to the hybridization medium can either be carried out simultaneously with the introduction of the solution containing the metal nanoparticles, or in succession therewith (either with or without a delay of up to about 15 minutes).

Because the colorimetric assay can be detected by naked eye observation, a user can either examine the hybridization solution for a detectable change in color or the assay can be carried out in parallel with one or more controls (positive or negative) that replicate the color of a comparable solution containing aggregated metal nanoparticles (negative control) and/or a comparable solution containing substantially non-aggregated metal nanoparticles (positive control).

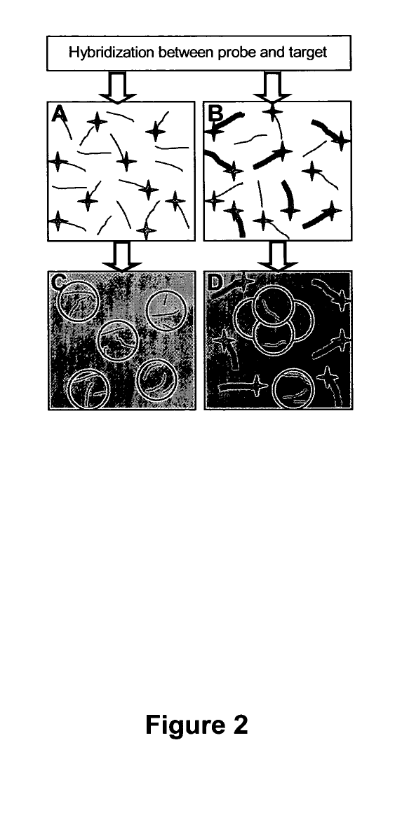

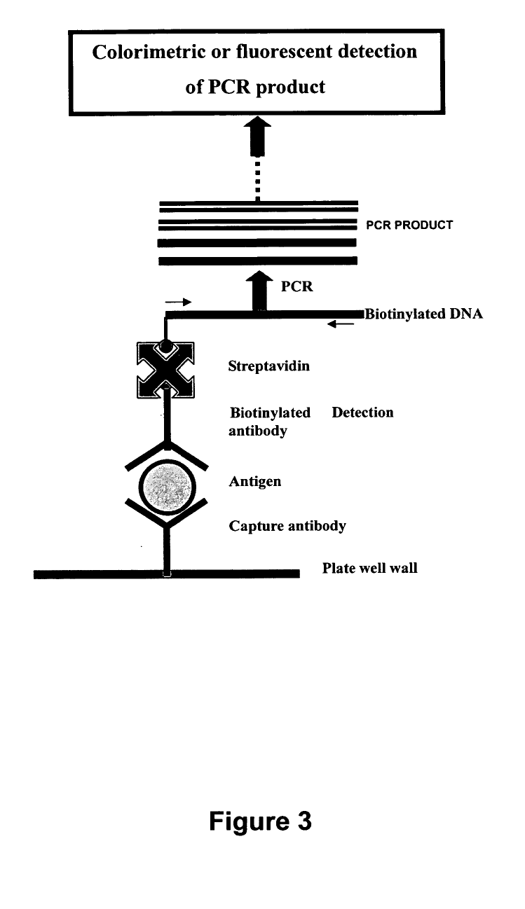

In a fluorimetric assay, the optical property of the hybridization solution is the fluorescence spectrum or the magnitude of a fluorescence peak by a fluorophore. The photoluminescent property of the fluorophore label is detected after the hybridization procedure is allowed to proceed in the presence of the metal nanoparticles. Because non-hybridizing oligonucleotide probes, based on their size, will more rapidly associate electrostatically with the metal nanoparticles than longer nucleic acid molecules in the hybridization solution, the absence of hybridization (i.e., absence of the target) is indicated by substantial quenching of fluorescence by the fluorescent label when oligonucleotide probes electrostatically associated with one or more metal nanoparticles. Hybridization between the oligonucleotide and the target nucleic acid molecule (i.e., presence of the target) is indicated by a maintained photoluminescent property even after aggregation of the metal nanoparticles (which is achieved in the same manner as described above). These alternatives are illustrated in FIG. 2. The fluorimetric assay is particularly useful for high sensitivity, when the target of interest is only one or many nucleic acid strands in a sample, when quantification of the target nucleic acid is desired, or when the presence of multiple distinct target nucleic acid molecules are being simultaneously analyzed within the same hybridization solution (i.e., using multiple oligonucleotide probes each with a distinct fluorophore attached thereto). Detection of the fluorescence properties of the hybridization solution can be achieved using appropriate detection equipment as is known in the art (e.g., fluorescence microscope, photomultipliers, CCD cameras, photodiodes, etc.

Because the fluorimetric assay involves measuring fluorescence caused by the fluorophore(s) in the hybridization solution, a user can either examine the hybridization solution for the presence or absence of fluorescence. No controls are necessary.

Because the fluorimetric assay is highly sensitive to even small quantities and the photoluminescent properties can be detected with precise instrumentation, the fluorimetric assay lends itself to quantifying the amount of a target nucleic acid present in a test solution. One approach for quantifying the amount of target nucleic acid present in the test solution involved comparing the results from the test solution to the results obtained from two control solutions that each contain known but differing amounts of the target nucleic acid. Thus, measurements of the photoluminescent property are obtained from the test solution and the two control solutions. Based on the photoluminescence of each solution, it is possible to calculate the quantity of the target nucleic acid in the test solution relative to the quantity of the target nucleic acid present in the first and second control solutions. Alternatively, the quantity of the target nucleic acid in the test solution can be calculated using the measured optical property (from the test solution) and a calibration curve of measured optical (e.g., photoluminescence) properties versus quantity of target nucleic acid.

One of the important uses of the assays of the present invention is with one or more forms of PCR, as noted above. Because PCR can quickly amplify the total amount of nucleic acid in a sample, it is often used with hybridization-based detection procedures. One of the significant benefits of the present invention is that the assay can be performed using the hybridization medium employed in the thermocycler. The only requirement, however, is that the product of PCR (typically a double-stranded cDNA) must be denatured prior to introducing the metal nanoparticles. Specifically, the double-stranded cDNA can be denatured before or after introducing the oligonucleotide probe to the hybridization medium, but before introducing the metal nanoparticles. Failure to denature the double-stranded cDNA will preclude hybridization between any target nucleic acid, if present, and the oligonucleotide probe, resulting in a possibly false negative result. Alternative PCR procedures that achieve a single-stranded product can be used without denaturing the PCR product.

Another important use of the assays of the present invention is for detecting a single nucleotide polymorphism (“SNP”) in a target nucleic acid molecule. This is performed in slightly different manners depending on whether the colorimetric assay or the fluorimetric assay is to be performed.

Basically, the colorimetric assay is performed in parallel using a test solution and a control solution. The test hybridization solution contains a target nucleic acid molecule and at least one first single-stranded oligonucleotide probe having a nucleotide sequence that hybridizes to a region of the target nucleic acid molecule that may contain a SNP. The probe contains a nucleotide sequence that does not hybridize perfectly to the region containing the SNP (i.e., no base-pairing occurs with the SNP). The control hybridization solution contains the target nucleic acid molecule and at least one second single-stranded oligonucleotide probe including a nucleotide sequence that hybridizes perfectly to a region of the target nucleic acid molecule that does not contain a single-nucleotide polymorphism. Both the test and control hybridization solutions are then exposed to the metal nanoparticles, allowing any unhybridized probes in the hybridization solutions to electrostatically associate with the metal nanoparticles. Importantly, during this stage of the assay, the hybridization solutions are maintained at a temperature that is between the melting temperature of the at least one first single-stranded oligonucleotide probe and the melting temperature of the at least one second single-stranded oligonucleotide probe (which has a higher melting temperature because it is perfectly complementary). Depending on the assay being performed (colorimetric or fluorimetric ), a determination is made whether an optical property of the test and control hybridization solutions are substantially different. A substantial difference indicates the presence of the single nucleotide polymorphism in the target nucleic acid molecule.

In detecting SNPs, the first and second single-stranded oligonucleotide probes can possess the same nucleotide sequence (and be the same length) or a different nucleotide sequence. That is, the two oligonucleotide probes can hybridize to the same region of the target nucleic acids or different regions. If the latter, then the target nucleic acid molecule in the control solution is, e.g., a cDNA molecule that is known not to possess the particular SNP being detected in the test solution. If the former, then the hybridization region of the target nucleic acid molecule in the control solution is known to be stable and free of SNPs (i.e., contains a wild-type sequence). To enhance the difference between the melting temperatures of the two oligonucleotide probes with their respective targets, the oligonucleotide probe for the control assay can be longer or can possess a modified structure (e.g., modified bases, backbone, etc.) that enhances the stability between the probe and target.

The fluorimetric assay is performed substantially as described above, except that the temperature of the hybridization solution is measured when quenching of photoluminescence from the fluorescent label begins (i.e., the temperature is slowly reduced until quenching begins). The measured temperature represents the melting temperature between the probe and the target nucleic acid. This measured melting temperature is then compared to a known melting temperature of a perfectly complementary probe (this measurement can either be provided with a commercial kit or measured by performing the assay in parallel). A difference between the melting temperatures indicates the presence of the single nucleotide polymorphism in the target nucleic acid molecule.

Yet another important use of the assays of the present invention is for detecting the presence of a pathogen in a sample. Basically, a sample is obtained (e.g., tissue sample, food sample, water sample, etc.) and nucleic acid is isolated from the sample. Having isolated the nucleic acid, either RNA or DNA, an assessment can be made as to whether enough of the sample is present to afford detection using the assays or whether PCR or RT-PCR is necessary to amplify the isolated nucleic acid. Thus, amplification may or may not be necessary. For example, total RNA isolated from a sample may be of sufficient quantity to proceed without RT-PCR; whereas total DNA isolated from a sample may require amplification. Regardless, the assay of the present invention is performed and the optical property (color or fluorescence intensity) of the hybridization solution is measured or assessed to determine whether or not the single-stranded oligonucleotide probe has hybridized to the target nucleic acid, indicating presence of the pathogen.

Yet another important use for the assays of the present invention is for genetic screening. Basically, a sample is obtained from a patient and nucleic acid is isolated from the sample. Because genetic screening will typically involve DNA isolation and analysis, it will typically (though not necessarily) require amplification. Regardless, the assay of the present invention is performed and the photoluminescent property of the hybridization solution is measured or assessed to determine whether or not the single-stranded oligonucleotide probe has hybridized to the target nucleic acid, indicating presence of a genetic marker for a genetic condition, a hereditary condition (e.g., paternity, maternity, relatedness, etc.), or identifying an organism.

A further use of the assays of the present invention is detection of a protein or antibody in a sample. Immuno-PCR is a procedure that can afford cDNA amplification only if a targeted protein is present in a sample. Thus, the assays of the present invention can be coupled with the amplification detection procedure of immuno-PCR to confirm presence of the amplified cDNA in the hybridization medium and, thus, the target protein in a sample. Basically, a sample is obtained and immuno-PCR is performed using the sample, wherein the immuno-PCR results in amplification of a nucleic acid that is conjugated to a protein. Thereafter, the assays of the present invention are performed where the nucleic acid that is conjugated to the protein (or its complement) becomes the target of the colorimetric or fluorimetric assay of the present invention.

A further use of the assays of the present invention is quantifying the amount of amplified nucleic acid prepared by polymerase chain reaction (or similar amplified procedure). Basically, one or more, and preferably two or more fluorescently labeled oligonucleotide primers are provided that each have a nucleotide sequence capable of hybridizing to a nucleic acid molecule, or its complement, that us to be amplified. Amplification using the primers is carried out using any of a variety of known amplification procedures (such as polymerase chain reaction) using a target nucleic acid molecule, and/or its complement, and the provided fluorescently labeled oligonucleotide primers. Thereafter, the fluorimetric method of the present invention is performed on a sample obtained after the amplification procedure has been performed. The level of fluorescence detected from the sample indicates the amount of primer that has been incorporated into an amplified nucleic acid molecule. As amplification continues (and incorporated more of the primers into longer, amplified sequences), the amount of fluorescence from a given sample should increase due to the reduced rate at which longer nucleic acid electrostatically associate to the metal nanoparticles. Unextended primers, on the other hand, will rapidly associate with the metal nanoparticles, which results in quenching of fluorescence by labels attached thereto.

A further aspect of the present invention relates to one or more types of kits that can be used to practice the assays of the present invention. The kits can include, among other components, a first container that contains a colloidal solution of metal nanoparticles, and a second container that contains an aqueous solution containing at least one single-stranded oligonucleotide probe having a nucleotide sequence that is substantially complementary to a target nucleic acid molecule. Depending on the assay to be performed (colorimetric or fluorimetric), the oligonucleotide probe in the second container may or may not be conjugated to a fluorescent label of the types described above. With fluorimetric assays and the ability to discriminate between multiple targets, the second container can optionally contain additional oligonucleotide probes (directed to the same or different target nucleic acid molecules), each having a distinct fluorescent emission pattern. In addition to the foregoing containers and components, containers containing control solutions, salt solutions for the colorimetric assay, and various instructions can also be provided.

### EXAMPLES

The following examples are provided to illustrate embodiments of the present invention but they are by no means intended to limit its scope.

### Materials and Methods for Example 1

A colloidal solution of gold nanoparticles of about 13 nm diameter synthesized via citrate reduction of HAuCl₄ (Grabar et al., Anal. Chem. 67:735-743 (1995), which is hereby incorporated by reference in its entirety) was used. The concentration of the colloidal solution was typically 17 nM. Lyophilized oligonucleotide sequences and their complements were purchased from MWG Biotech (High Point, N.C.) and dissolved in 10 mM phosphate buffer solution. Typically, attempted hybridization of the probe and the target was conducted at room temperature for 5 minutes in 10 mM phosphate buffer solution containing 0.3 M NaCl. Specific salt concentrations vary with experiment and are stated in the figure captions. Following the trial hybridization, the trial solution was mixed with gold colloid and immediately followed by addition of salt/buffer solution.

Samples were placed in quartz cuvettes with 5 mm path length to record absorption spectra using a Perkin Elmer UV/VIS/NIR spectrometer Lambda 19 with water as a reference. For fluorescence spectra and intensities versus time, dye labeled oligonucleotides purchased from MWG Biotech (High Point, N.C.) were used. Solutions in quartz cells with 1 cm path length were studied on a Jobin-Yvon Fluorolog-3 spectrometer with front face collection geometry and 4 nm resolution. Resonance Raman spectra were taken on these dye labeled oligonucleotides with steady state 532 nm excitation and detection by an Ocean Optics CCD array with a holographic notch filter to reject Rayleigh scattering. The resolution was approximately 10 cm⁻¹. Photographs were taken with a Canon S-30 digital camera.

### Example 1

### Gold Nanoparticles Preferentially Adsorb Single-Stranded Nucleic Acid Rather Than Double-Stranded Nucleic Acid

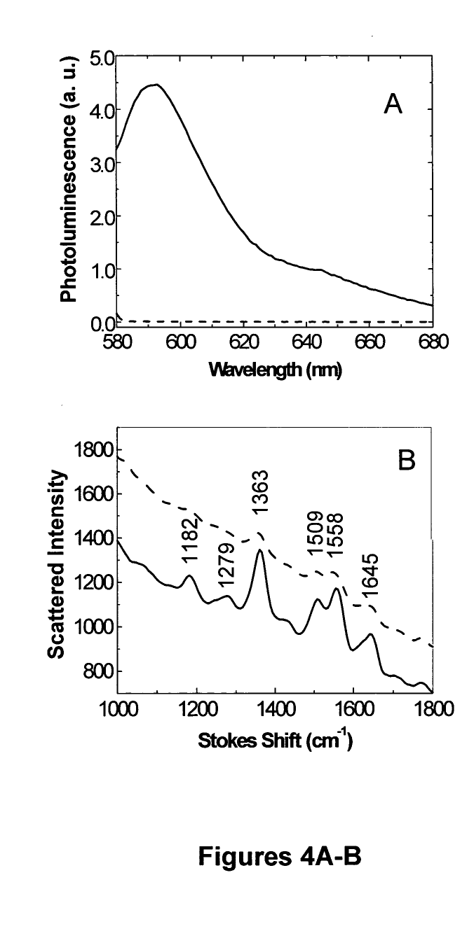

Direct evidence for the preferential interaction between dye-tagged ss-DNA and gold nanoparticles is illustrated in FIGS. 4A-B. The fact that dye-tagged ss-DNA adsorbs on the gold while ds-DNA does not can be seen through the effects of adding colloidal gold to solutions containing either dye-tagged ss-DNA or dye-tagged ds-DNA. In the case of dye tagged ss-DNA, quenching of the dye photoluminescence and enhancement of resonant Raman scattering from the dye were observed. Both of these require intimate contact between the dye and the gold since they are effects of electronic interactions with the gold plasmons.

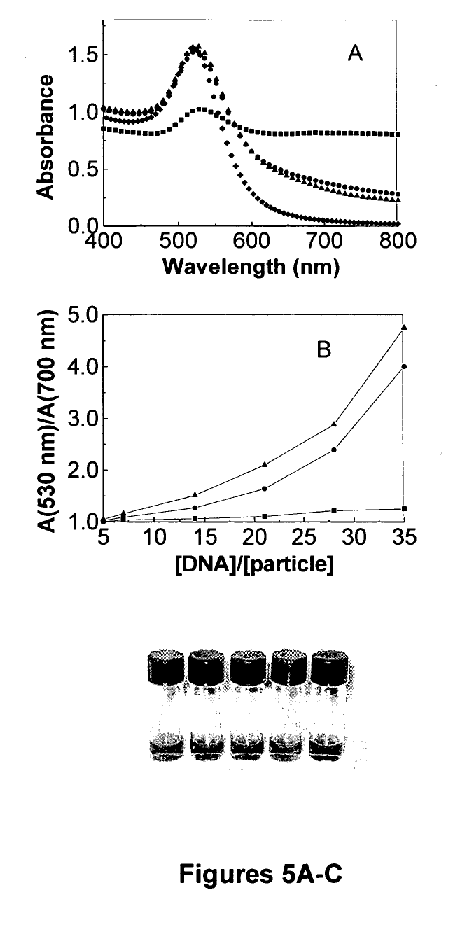

FIG. 5A presents spectra of the colloid prior to and after addition of ss-DNA or ds-DNA and salt/buffer solution. Ordinarily, exposure to salt screens the repulsive interactions and causes colloid aggregation (Hunter, Foundations of Colloid Science, Oxford University Press Inc., New York (2001); Shaw, Colloid and Surface Chemistry, Butterworth-Heinemann Ltd., Oxford (1991), which are hereby incorporated by reference in their entirety). Apparently, the adsorption of the ss-DNA based on the gold nanoparticles additionally stabilizes the colloidal gold particles against aggregation when salt is introduced. Thus, solutions with adequate quantities of ss-DNA prevent aggregation and the gold colloid remains pink while solutions with ds-DNA do not affect the aggregation and the solutions turn blue. Presumably, this has to do with a redistribution of charge that makes the surface appear more negatively charged. The Raman studies suggest that the ss-DNA does not replace the citrate ions.

FIG. 5B illustrates a condensed form of the same data for two ss-DNA sequences and documents how the color depends on the amount of ss-DNA. Remarkably, solutions with only a few ss-DNA per gold nanoparticle have distinctly different absorption spectra in spite of the fact that the surface area of the nanoparticles is sufficient to accommodate several hundred ss-DNA 24-mers. With enough ss-DNA, the colloid retains a pink coloring while hybridization of the trial solution to form ds-DNA leads to a bluish colloid (FIG. 5C). From a practical point of view, this allows the design of an assay to determine whether a given sample contains single stranded or double stranded DNA along the lines of the protocol depicted in FIG. 1. An extremely important feature of the method is that hybridization can be done with label free oligonucleotides under optimized conditions (pH, salt, and buffer concentrations) and is completely independent of the detection step. Also investigated is what happens with concentration mismatches between target and probe by using solutions where their ratio is varied from 0 to 1. The results (FIG. 5C) prove the technique to be surprisingly robust in its ability to detect the presence of the target. Calibrated colorimetric measurements could be used to determine the amount of target quantitatively.

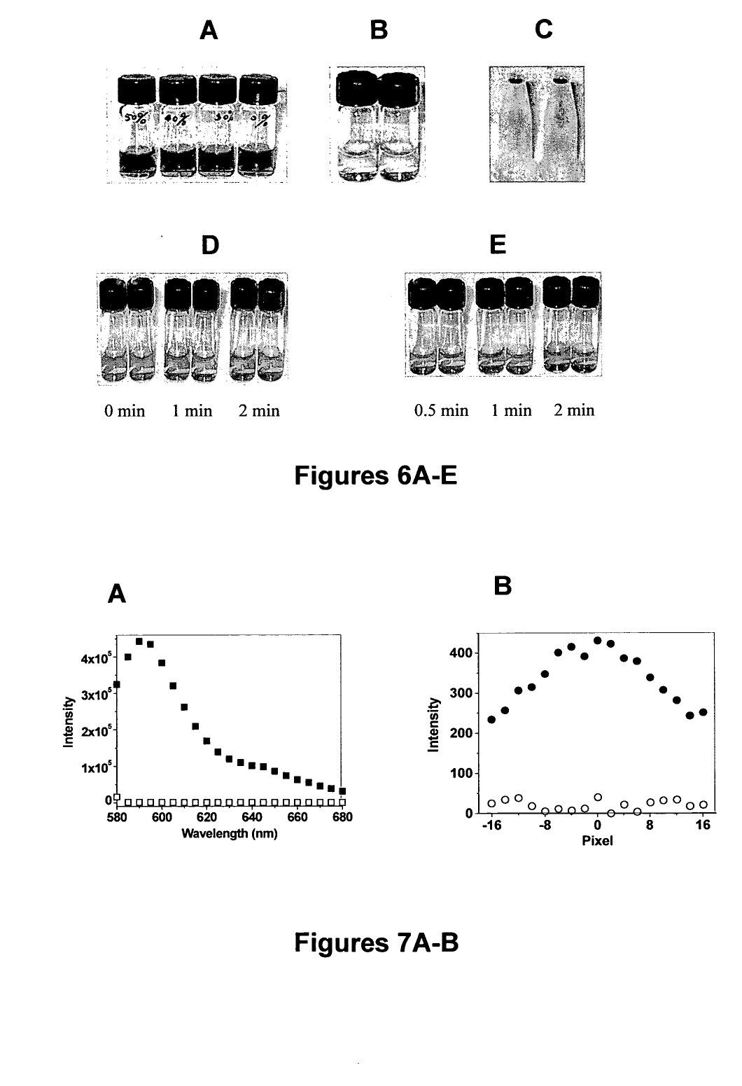

Similarly, one can consider the case where the analyte solution contains a mixture of oligonucleotide sequences as might occur in products of polymerase chain amplification, where primers and other fragments are present (Rolfs et al., PCR: Clinical Diagnostics and Research, Springer-Verlag, Berlin Heidelberg (1992), which is hereby incorporated by reference in its entirety). FIG. 6A illustrates the result for a mixed oligonucleotide analyte with various fractions of target sequence and it is clear that as little as 30% target is easily detected. A situation similar to concentration mismatch occurs when the target and probe sequences are complementary but have different lengths. In that case, one could imagine that some of the hybridized chain appears to have the electrostatic properties of ss-DNA while other portions appear double stranded. Qualitatively, the results are similar to those with perfect length match and even hybridized probe and target strands with relatively large length differences (on the order of 5-10 base pairs) behave as double stranded.

The extraordinarily high extinction coefficient of gold nanoparticles (Doremus, J. Chem. Phys. 40:2389-2396 (1994), which is hereby incorporated by reference in its entirety) makes the colorimetric method extremely sensitive. At 17 nM concentration (Grabar et al., Anal. Chem. 67:735-743 (1995), which is hereby incorporated by reference in its entirety), a 1 cm path length provides optical densities near unity. Empirically, it is easy to visually identify the colour in 5 μL droplets that contain less than 100 femtomoles of gold particles. FIG. 5B illustrates that ss-DNA concentrations only slightly greater than the nanoparticle concentration are sufficient to stabilize the colloid against aggregation when exposed to salt. Consequently, one would expect to be able to differentiate between amounts of ss- and ds-DNA of order 100 femtomoles without instrumentation. Even though adsorption of only one or two ss-DNA strands per nanoparticle covers very little of the gold's surface area, it appears to add net negative charges that are distributed around the nanoparticle through rearrangement of charges in the citrate coating. Consistent with the above reasoning, target concentrations of 4.3 nM (FIG. 6B) or total amounts of target as low as 60 femtomoles (FIG. 6C) produce easily visible differences. Utilizing an absorption spectrometer to evaluate color should produce at least an order of magnitude improvement in sensitivity and use of a null method for measuring absorption, such as photo-thermal deflection, would still further enhance sensitivity (Jackson, Applied Optics 20:1333-1344 (1981), which is hereby incorporated by reference in its entirety).

The method is easily adapted to identifying single base pair mismatches between probe and target as is essential for detection of biologically important single nucleotide polymorphisms (Rolfs et al., PCR: Clinical Diagnostics and Research, Springer-Verlag, Berlin Heidelberg (1992), which is hereby incorporated by reference in its entirety). Utilized was the fact that the kinetics of ds-DNA dissociation into ss-DNA fragments depend on the binding strength (Owczarzy et al., Biopolymers 44:217-239 (1997); Santalucia et al., J. Am. Chem. Soc. 113:4313-4322 (1991), which are hereby incorporated by reference in their entirety) and are therefore faster for mismatched ds-DNA (ds′-DNA) than for perfectly matched ds-DNA. The ds-DNA from the trial solution was allowed to dehybridize briefly in water without salt before adding gold colloid and the salt/buffer solution. An obvious color difference was observed between perfectly matched (5′-TAC GAG TTG AGA ATC CTG AAT GCG-3′ (SEQ ID NO: 1) and its complement) and single base pair mismatched ds-DNA segments (5′-TAC GAG TTG AGA ATC CTG AAT GCG-3′ (SEQ ID NO: 2) and 5′-CGC ATT CAG GCT TCT CAA CTC GTA-3′ (SEQ ID NO: 3) waiting 2 minutes before performing the assay (FIG. 6D). While dehybridization can also be done in the gold colloid solution simply by delaying the introduction of the buffer/salt solution, the ds-DNA is found more stable in the colloid solution than in water, and there is no significant dehybridization as determined by the assay after 10 minutes in gold colloid. A single base pair mismatched DNA segment showed obvious dehybridization after 5 minutes. Subjecting the mixture of oligonucleotide solution and gold colloid to ultrasound for 1 or 2 minutes before mixing with buffer/salt solution accelerated the dehybridization and also gave excellent contrast between ds-DNA and ds′-DNA (FIG. 6E).

It has been demonstrated that ss-DNA and ds-DNA have different propensities to adsorb on gold nanoparticles due to their electrostatic properties. This has been used to design an oligonucleotide recognition assay that uses only commercially available materials, takes less than ten minutes, requires no detection apparatus, is sensitive to single base mismatches, and is reasonably tolerant of concentration or length mismatches. The assay described has additional benefits beyond its speed and simplicity. Because of the ability to exploit the electrostatic properties of the DNA, hybridization is separated from detection so that the kinetics and thermodynamics of DNA binding are unperturbed by steric constraints associated with probe functionalized surfaces. In addition, the assay is homogeneous as it occurs exclusively in the liquid phase, a feature that makes it easy to automate using standard robotic manipulation of microwell plates. The ability to differentially adsorb ss-DNA onto the gold particles can also form the basis for a sensitive assay based on fluorescence that still avoids tagging of the analyte. With fluorescent dyes incorporated onto the probe strands, the fluorescence of the ss-DNA can be selectively quenched as in FIG. 4A since it forces the dye to be near the gold nanoparticles where the fluorescence is quenched (Dubertret et al., Nature Biotechnol. 19:365-370 (2001); Du et al., J. Am. Chem. Soc. 125:4012-4013 (2003), which are hereby incorporated by reference in their entirety). If the tagged probe ss-DNA binds the target, however, the ds-DNA does not adsorb on the gold and the fluorescence persists.

### Materials and Methods for Examples 2-6

Gold particles with 13 nm diameter were synthesized by reduction of HAuCl₄ (Gradar et al., Anal. Chem. 67:735-743 (1995), which is hereby incorporated by reference in its entirety). Briefly, 500 mL of 1 mM HAuCl₄ was brought to a rolling boil with vigorous stirring. 50 mL of 38.8 mM sodium citrate was quickly added to the solution, and boiling was continued for 10 min. The heating mantle was then removed and the stirring was continued for an additional 15 minutes.

All oligonucleotides were purchased from MWG Biotech, Inc. (High Point, N.C.) without further purification. Probes hybridize with targets in 10 mM phosphate buffer solution with 0.3 M NaCl for more than 5 minutes at room temperature or proper temperature.

The probes and targets employed in Example 2 are as follows:
Rhodamine red-labeled probe: AGGAATTCCATAGCT (SEQ ID NO: 25); and
Target nucleic acid: AGCTATGGAATTCCT (SEQ ID NO: 26).

The probes and targets employed in Examples 3 and 4 are as follows:
Rhodamine red-labeled probe: AGGAATTCCATAGCT (SEQ ID NO: 27);
Complementary Target A: ACTAGGCACTGTACGCCAGCTATGGAATTCCTT AGCTATGAGATCCTTCG (SEQ ID NO: 28);
Complementary Target B: GTTAGCTATGAGATCCTTCGTAGGCACTGTACGC CAGCTATGGAATTCCT (SEQ ID NO: 29); and
Noncomplementary Target C: TGTGTTGAACCTGGTGAAGTTGTAATCTGGAA CTTGTTGAGCAGAGGTTC (SEQ ID NO: 30).

The probes and targets employed in Example 5 are as follows:
Rhodamine red-labeled probe: AGGAATTCCATAGCT (SEQ ID NO: 31);
Complementary Target A′: ACTAGGCACTGTACGCCAGCTATCGAATTCCT TAGCTATGAGATCCTTCG (SEQ ID NO: 32); and
Complementary Target B′: GTTAGCTATGAGATCCTTCGTAGGCACTGTAC GCCAGCTATCGAATTCCT (SEQ ID NO: 33).

The probes and targets employed in Example 6 are as follows:
Rhodamine red-labeled probe 1: CTGAATCCAGGAGCA (SEQ ID NO: 34);
Complementary Target 1: the complement of probe 1;
Cy5-labeled probe 2: TAGCTATGGAATTCCTCGTAGGCA (SEQ ID NO: 35);
Complementary Target 2: the complement of probe 2; and
Non-complement target: ATGGCAACTATACGCGCTAC (SEQ ID NO: 36).

A fraction of hybridized solution was added to 500 μL of 17 nM gold colloid solution, and an additional 500 μL of the 0.1 M saline 10 mM phosphate buffer solution was added if without specific illustration. The fluorescence of this mixture was recorded immediately using either a fluorimeter, or a fluorescence microscope and camera. Fluorescence spectra were measured on a fluorimeter with excitation at 570 nm, and emission range from 585 to 680 nm, with slits set for 4 nm bandpass unless specific illustration was given. Fluorescence images were recorded with a fluorescence confocol microscope equipped with notch filter and narrow bandpass interference filter. Fluorescence was excited by a 532 nm laser source.

### Example 2

### Differential Fluorescence Quenching of Dye-Tagged Single-Stranded DNA and Double-Stranded DNA

DNA oligonucleotides labeled with rhodamine red fluorescent dye covalently attached at the 5′ end were used as probes. Several microliters of μM solutions of probe were exposed to the target sequences for trial hybridization in 10 mM phosphate buffer with 0.3 M NaCl. The hybridization solutions were added to colloidal gold suspensions and additional phosphate buffer saline solution was added to assist in stabilizing ds-DNA.

FIG. 7A illustrates the result of a measurement comparing the photoluminescence from trial solutions with complementary and non-complementary targets. Fluorescence contrast larger than 100:1 was observed because unhybridized probes efficiently adsorb on the gold nanoparticles so that their fluorescence is quenched. The adsorption mechanism is entirely electrostatic, as discussed in Example 1 above. The adsorption and concomitant fluorescence quenching are irreversible.

Addition of the trial hybridization solutions and salt to the gold colloid eventually cause aggregation of the colloid. The latter leads to precipitation so that the nanoparticles are no longer an effective quencher of the probe fluorescence. It is possible to protect the colloid against aggregation under conditions with sufficient salt to satisfy the duplex by using unrelated ss-DNA strands to stabilize the colloid. However, the data of FIG. 7A illustrates that this is not necessary as long as the fluorescence measurements are made within about 15 minutes.

Since relatively large volumes of solution are required for a typical fluorimeter, it is not practical to assess the sensitivity of the method using the same measurement protocol. FIG. 7B illustrates measuring the fluorescence of a very small aliquot of the solution containing only 0.1 femtomoles of target and this is easily detected with a fluorescence microscope and camera. Since the method is essentially a null method, it stands to reason that it can be used in a relatively straightforward fluorescence detection down to fewer than 10 copies of target oligonucleotide (0.1 attomole) (Cao et al., Science 297:1536-1540 (2002), which is hereby incorporated by reference in its entirety).

### Example 3

### Application to Long Target Sequences

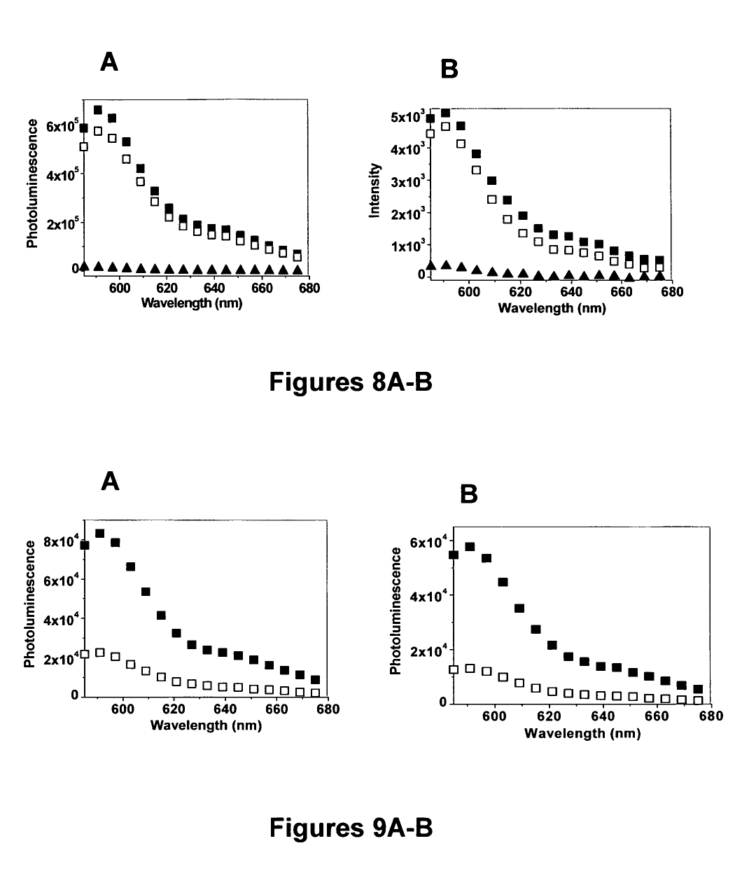

For genomic analysis, it is desirable to detect specific sequences on much longer DNA targets than synthesized oligonucleotides. These could be derived directly from clinical samples or from samples that have been amplified using PCR. FIG. 8A is a proof of principle for detecting matches to parts of long targets. In spite of the fact that large portions of the target remain single stranded and will presumably have the electrostatic properties of ss-DNA, the assay can be used to determine whether these long targets contain sequences complementary to short dye-tagged probes. The reason adsorption and quenching are not observed in this case is that long ss-DNA sequences adsorb on the gold nanoparticles at a much slower rate, as noted in Example 7 herein. Thus, the technique is most practical when short dye-tagged probes (<25 mers) are used.

### Example 4

### Application to Mixtures of Target Sequences

Because the only requirement of the assay is that ss-DNA probes that do not hybridize to a target sequence in the analyte adsorb on gold and are quenched, the only constraint is that the amount of colloidal gold must be adequate to adsorb all of the probe DNA. Therefore, the assay can work to determine whether target strands are present even in complex mixtures of DNA oligonucleotides as demonstrated by the data of FIG. 8B. In that case, 1% complementary target was mixed with 99% non-complementary target to verify the presence of the target sequence. The tolerance of the assay to mixtures, along with its sensitivity, provides the potential for it to be used without target amplification by PCR.

### Example 5

### Single Base Mismatch Detection

It is simple to adapt the technique to detect single base mismatches by introducing a perfectly matched control and comparing the two with a stringency test. For illustrative purposes, two different target sequences that differ by a single base were used. One of these is perfectly matched to the dye-tagged probe. The only procedural difference is that, before introducing the two trial hybridization solutions to gold colloid, they are each held for 5 minutes at 46° C., a temperature above the melting temperature for the mismatch and below that for the perfect match. The mismatched strand dehybridizes, thereby releasing single stranded probe whose fluorescence can be quenched. The sample with a mismatch therefore exhibits much less photoluminescence than the perfectly matched target. FIG. 9 shows the detection by one long target complementary to the probe in the middle portion and another long target complementary to the probe at one end. This procedure should be applicable to rapid detection of single nucleotide polymorphisms in genomic DNA, an exciting prospect for eliminating time-consuming and expensive gel sequencing procedures that are currently the standard protocol (Rolfs et al., PCR: Clinical Diagnostics and Research, Springer-Verlag, Berlin Heidelberg (1992), which is hereby incorporated by reference in their entirety). In practice, of course, one would use two different probe strand sequences and compare probes complementary to the wild type sequence to ones with single base mismatches at the targeted locations.

### Example 6

### Simultaneous Multiple Target Detection

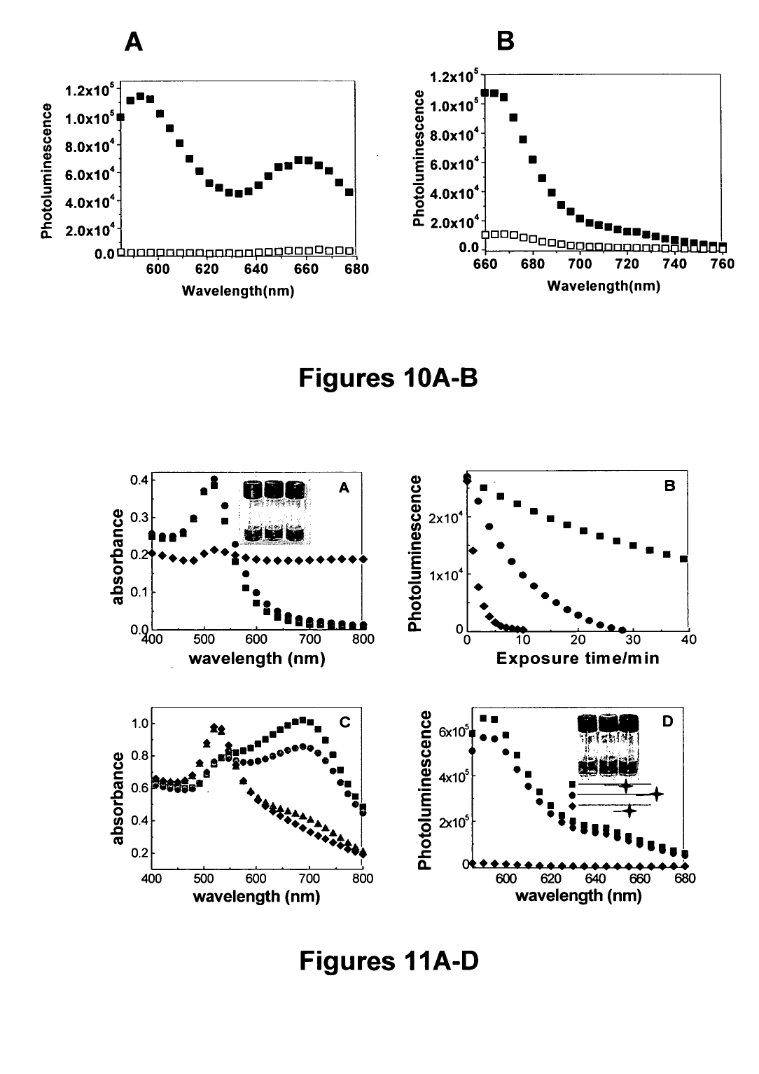

The differential quenching assay can also be multiplexed to simultaneously look for several sequences on a single target or for several targets. FIG. 10 illustrates this where two different probes with two different dyes are hybridized with a mixture of targets. If spectroscopic detection is used, it is plausible to imagine expanding this approach to 5 or 6 targets with conventional dyes and even more with semiconductor nanoparticle fluorophores that have spectrally sharp emission. This, of course, presumes that these do not perturb the essential electrostatics that is the basis of the method.

In summary, these experiments demonstrate a simple assay for DNA sequence recognition based on the difference in electrostatic properties of ss-DNA and ds-DNA. For certain salt concentrations, ss-DNA adsorbs on citrate-coated gold nanoparticles while ds-DNA does not and this fact can be exploited to differentially quench fluorescence of a dye-tagged ss-DNA probe. The method requires no target modification, uses only commercially available materials, works for analytes with mixtures of oligonucleotides, and can be applied to detection of single base mismatches. Perhaps the most attractive feature of the approach is its speed. The entire assay can be completed in less than 10 minutes because the hybridization step occurs in solution under optimized conditions and is separated from the detection step. A sensitivity to less than 0.1 femtomole of DNA oligonucleotides has been demonstrated, but, because the method is nearly a null method and relies on fluorescence detection, it is probably possible to improve this by several orders of magnitude. It is believed that the method has enormous promise for applications to pathogen detection, clinical analysis of SNPs, and biomolecular research.

### Materials and Methods for Examples 7-9

All synthesized oligonucleotides were purchased from MWG Biotech, Inc. (High Point, N.C.), and used without further purification.

Colloidal solutions of gold nanoparticles were synthesized according to the procedure described in Grabar et al., Anal. Chem. 67:735-743 (1995), which is hereby incorporated by reference in their entirety). Briefly, 250 mL of 1 mM HAuCl₄ (Alfa Aesar, Ward Hill, Mass.) was heated to its boiling point while stirring. 25 mL of 38.8 mM sodium citrate (Alfa Aesar, Ward Hill, Mass.) was added quickly to the boiling solution, while continuing to boil and stir the solution for another 15 minutes. The solution was cooled to room temperature and can be stored indefinitely for use.

All photographs in this work were recorded with a Canon PowerShot S30 digital camera. Absorption spectra were recorded on a Perkin Elmer UV/IS/NIR spectrometer Lambda 19. Quartz cells with 2 mm or 5 mm path length were used and water was used as reference. Fluorescence spectra and intensities versus time were recorded on a Jobin-Yvon Fluorolog-3 spectrometer with excitation at 570 nm and emission at 590 nm, each with slits set for 4 nm bandpass. Quartz cells with 1 cm path length and front face collection were used for the fluorescence measurements.

### Example 7

### Effects of Oligonucleotide Probe Length and Temperature on Adsorption of ss-DNA to Gold Nanoparticles

To study effects of ss-DNA on gold nanoparticle aggregation, 300 μL gold colloid was mixed with 300 picomole 24 mer ss-DNA (5′-TGC CTA CGA GGA ATT CCA TAG CTA-3′ (SEQ ID NO: 4)) in 10 μL of 10 mM PBS containing 0.2 M NaCl, and then 100 μL of 10 mM PBS containing 0.2 M NaCl was added. For comparison, 100 μL deionized water was mixed with 100 μL of 10 mM PBS containing 0.2 M NaCl with 300 μL gold colloid, respectively. Absorption spectra were recorded with 2 mm pathlength cells and photographs of the mixtures were taken. The spectra are stable with time.

To investigate sequence length dependent adsorption of ss-DNA to gold nanoparticles, 2 μL (2 μM) ss-DNAs with rhodamine red tags at the 5′ end were added to 1000 μL of 13 nm gold colloid. The ss-DNA sequences were 10 mer (5′-CAG GAA TTC C-3′ (SEQ ID NO: 5)), 24 mer (5′-TAG CTA TGG AAT TCC TCG TAG GCA-3′ (SEQ ID NO: 6)), and 50 mer (5′-GAA CCT CTG CTC AAC AAG TTC CAG ATT ACA ACT TCA CCA GGT TCA ACA CA-3′ (SEQ ID NO: 7)). The fluorescence intensity versus time was recorded on the fluorimeter.

To study the temperature dependence of ss-DNA adsorption, mixtures of 2 μL (100 μM) 50 mer ss-DNA and 300 μL of 13 nm gold colloid were heated to 22° C., 45° C., 70° C., and 95° C. for two minutes, respectively. Solutions of 300 μL of 10 mM PBS at 22° C. containing 0.2 M NaCl were added immediately and absorption spectra were measured with 5 mm pathlength cells.

To study the adsorption of short and long ss-DNA mixtures, 4 μL of 2 μM rhodamine red labeled 15 mer (5′-AGG AAT TCC ATA GCT-3′ (SEQ ID NO: 8)) was mixed with each of three different 50 mers (sequences infra) in 10 mM PBS containing 0.3 M NaCl (4 μL at 2 μM concentration) for trial hybridization.
5′-AC TAG GCA CTG TAC GCC AGC TAT GGA ATT CCT TAG CTA TGA GAT CCT TCG-3′ (SEQ ID NO: 9) complementary to the 15 mer at middle;
5′-GT TAG CTA TGA GAT CCT TCG TAG GCA CTG TAC GCC AGC TAT GGA ATT CCT-3′ (SEQ ID NO: 10) complementary to the 15 mer at end.
5′-TGT GTT GA ACCT GGT GAA GTT GTA ATC TGG AAC TTG TTG AGC AGA GGT TC-3′ (SEQ ID NO: 11) non-complementary to the 15 mer.
   After 5 minutes for hybridization, each solution was mixed with 1 mL of 13 nm gold colloid and 0.4 mL additional 10 mM PBS containing 0.1 M NaCl and the resulting fluorescence spectrum was recorded on fluorimeter. Color photographs of the mixtures of 300 μL gold colloid, 6 μL (20 μM) hybridized DNA solution and 300 μL of 10 mM PBS containing 0.2 M NaCl taken with a Canon S-30 camera without unlabeled 15 mer of the same sequence.

The color of gold colloid is very sensitive to the degree of aggregation of nanoparticles in suspension (Quinten et al., Surf. Sci. 172:557 (1986); Lazarides et al., J. Phys. Chem. B 104:460 (2000); Storhoff et al., J. Am. Chem. Soc. 122:4640-4650 (2000), which are hereby incorporated by reference in their entirety), and the aggregation can be easily induced with electrolytes such as salt (Hunter, Foundations of Colloid Science, Oxford University Press Inc., New York (2001); Shaw, Colloid and Surface Chemistry, Butterworth-Heinemann Ltd., Oxford (1991), which are hereby incorporated by reference in their entirety). This phenomenon can be easily monitored by absorption spectroscopy or by visual observation. Gold nanoparticles (13 nm in diameter) in aqueous solution are stabilized against aggregation by a negatively charged coating of citrate ions (Bloomfield et al., Nucleic Acids: Structures, Properties, and Functions, University Science Books, Sausalito, Calif. (1999), which is hereby incorporated by reference in its entirety). As individual particles, gold nanoparticles have surface plasma resonance absorption peak at 520 nm (FIG. 11A: red) and appear pink (FIG. 11A, inset: left vial). Immediate aggregation of the gold nanoparticles occurs when enough salt is added to screen the electrostatic repulsion between the ion-coated gold nanoparticles. The result is a broad featureless absorption spectrum (FIG. 11A: blue) and blue-gray color (FIG. 11A, inset: middle vial) characteristic of the surface plasma resonance of gold nanoparticle aggregates (Quinten et al., Surf Sci. 172:557 (1986); Lazarides et al., J. Phys. Chem. B 104:460-467 (2000); Storhoff et al., J. Am. Chem. Soc. 122:4640-4650 (2000), which are hereby incorporated by reference in their entirety).

It was found that the salt no longer causes aggregation of the gold nanoparticles if enough ss-DNA is added to the gold colloid before addition of the salt that would otherwise cause aggregation. Under these circumstances, the gold colloid retains its absorption spectrum and color (FIG. 11A: green and inset: right vial). The reason for the stabilization of the colloid is that the oligonucleotides adsorb and add negative charges to the gold nanoparticles that enhances their repulsion. This assertion is confirmed by fluorescence quenching experiments using rhodamine red-tagged ss-DNA (FIG. 11B). When the oligonucleotide adsorbs to the gold nanoparticle, the attendant proximity of the dye to the gold leads to fluorescence quenching (Maxwell et al., J. Am. Chem. Soc. 124:9606-9612 (2002); Dubertret et al., Nat. Biotech. 19:365-370 (2001), which are hereby incorporated by reference in their entirety). The fluorescence quenching experiments also show that the adsorption rate depends on sequence length, with shorter sequences sticking much more rapidly to the gold nanoparticle (FIG. 11B). In addition, it is found that increasing temperature also results in faster adsorption (FIG. 11C). Both the ss-DNA adsorption on gold nanoparticle and the gold nanoparticle aggregation inferred from the data in FIGS. 11A-D are irreversible.

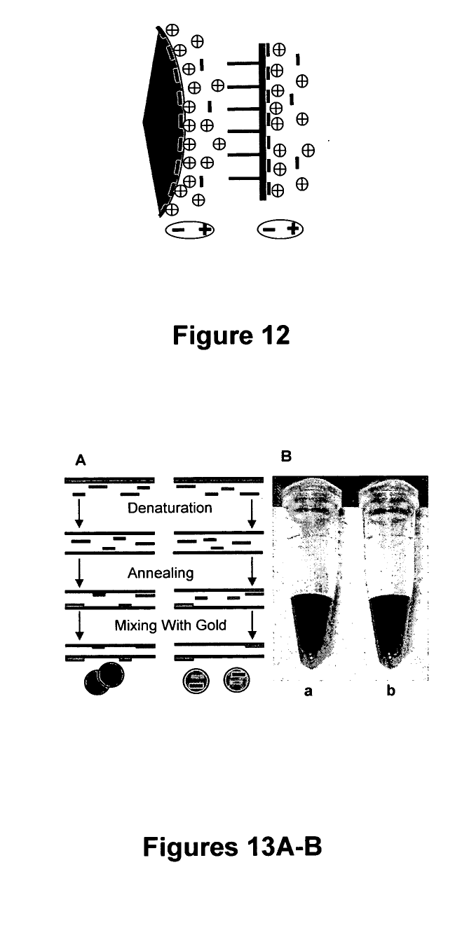

The adsorption of ss-DNA on negatively charged gold nanoparticles is contrary to the conventional wisdom (Maxwell et al., J. Am. Chem. Soc. 124:9606-9612 (2002); Graham et al., Angew. Chem. Int. Ed. 39:1061 (2000), which are hereby incorporated by reference in their entirety) since, in its native configuration, ss-DNA is coiled so that the hydrophilic negatively charged phosphate backbone is most exposed to the aqueous solution (Bloomfield et al., Nuclei Acids: Structures, Properties, and Functions, University Science Books, Sausalito, Calif. (1999), which is hereby incorporated by reference in its entirety). The fact that ss-DNA sticks to gold nanoparticles, as well as the dependence on sequence length and temperature, can be explained with a simple picture derived from the theory of colloid science (Hunter, Foundations of Colloid Science, Oxford University Press Inc., New York (2001); Shaw, Colloid and Surface Chemistry, Butterworth-Heinemann Ltd., Oxford (1991), which are hereby incorporated by reference in their entirety). Both the gold nanoparticle and the ss-DNA attract counterions from the solution and are well described by electrical double layers as depicted schematically in FIG. 12. In every case, there are attractive Van der Waals forces between the oligonucleotide and the nanoparticle. The electrostatic forces are due to dipolar interactions and depend on the configuration and orientation of the ss-DNA. When transient structural fluctuations permit short segments of the ss-DNA to uncoil and the bases face the gold nanoparticle, attractive electrostatic forces cause ss-DNA to adsorb irreversibly to the gold. The requisite fluctuations are more prevalent in short sequences since there is less of the chain remaining to enforce the coiled morphology. Hence, short ss-DNA oligonucleotides adsorb more quickly. Similarly, increases in temperature facilitate fluctuations that expose the bases and unwind the coiled structure to make the adsorption faster. Increases in temperature will also serve to break secondary structure in longer DNA chains thereby making the geometry of FIG. 12 more easily accessible.

The length dependent adsorption can be exploited to develop an assay appropriate to detection of PCR amplified DNA sequences that are typically several hundred base pairs in extent (Reed et al., Practical Skills in Biomolecular Sciences, Addison Wesley Longman Limited, Edinburgh Gate, Harlow, England (1998); Walker et al., Molecular Biology and Biotechnology, The Royal Society of Chemistry, Thomas Graham House, Cambridge, UK (2000), which are hereby incorporated by reference in their entirety). Short oligonucleotide “probes” can be designed with the idea that, when these are hybridized to the long strands, they will not adsorb rapidly on gold nanoparticle. They will therefore be unable to prevent salt-induced aggregation and the attendant color changes when there is a sequence match between the probe and part of the long strand. Alternatively, if the short probes are fluorescently labeled, their fluorescence will be quenched by adsorption on the gold nanoparticle unless they are “tied up” by hybridization to the long target strand. FIG. 11D illustrates the proof of principle for each of these assays with synthesized 50 base oligonucleotide targets and rhodamine-labeled 15 base probe.

### Example 8

### Detection of PCR-Amplified Target cDNA

Genomic DNA obtained from Dr. Ming Qi of the University of Rochester Medical Center was used as PCR template. Primers were synthesized oligonucleotides 5′-CCT GGG CAT TAA GGT TCC-3′ (SEQ ID NO: 12) (forward) and 5′-TGG GAT TCT TCG GCT TCT TC-3′ (SEQ ID NO: 13) (reverse). The specific region of KCNE1 gene indicative of long QT syndrome was amplified in Promega PCR master mix (Promega, Madison, Wis.) with Tag DNA polymerase for 5 min at 95° C.; 35 cycles of 30s at 95° C., 30s at 56° C. and 30s at 72° C.; 10 min at 72° C. and then held at 4° C., yielding 189 bp PCR product.

Following these model experiments, simple colorimetric assays have been designed that address the critical issues that arise in the analysis of PCR amplified DNA. First, one can ascertain whether the amplified DNA contains the desired sequence by evaluating hybridization with the probes. Second, it is straightforward to identify SNPs in the amplified sequences. All of the experiments are performed on PCR product obtained from a clinical diagnosis laboratory without further purification. The sequence probed derives from genomic DNA taken in patient studies of a fatal cardiac arrhythmia called long QT syndrome (Priori et al., J. Interv. Card. Electr. 9:93 (2003), which is hereby incorporated by reference in its entirety). This condition has been associated with a mutation in KCNE1 gene (Splawski et al., Circulation 102:1178-1185 (2000), which is hereby incorporated by reference in its entirety).

Current assays for point mutations on PCR amplified sequences involve time-consuming procedures, expensive instrumentation or both (Reed et al., Practical Skills in Biomolecular Sciences, Addison Wesley Longman Limited, Edinburgh Gate, Harlow, England (1998); Walker et al., Molecular Biology and Biotechnology, The Royal Society of Chemistry, Thomas Graham House, Cambridge, UK (2000); Rolfs et al., PCR: Clinical Diagnostics and Research, Springer-Verlag, Berlin Heidelberg (1992), which are hereby incorporated by reference in their entirety). The method takes less than ten minutes to verify amplification of the appropriate sequence and test for SNPs with the same thermal cycler used to do the PCR. To confirm amplification of the desired sequence, the protocol illustrated schematically in FIG. 13A was followed. Two ss-DNA probes were chosen with sequences complementary to the desired PCR product that have melting temperatures lower than the primers and add these to the PCR product solution. The PCR amplified ds-DNA is dehybridized at 95° C. to produce ss-DNA. These mixtures are annealed below the probe melting temperature so that the probes can hybridize with the PCR amplified sequence if it is present. At the same time, the unconsumed primers also bind to the PCR product since they have melting temperatures higher than those of the probes. As in the PCR process itself, competition for binding locations from rehybridization of the PCR amplified complement is negligible since it is slower for steric reasons. When gold colloid is exposed to this mixture, the salt in the hybridization solution causes immediate gold nanoparticle aggregation and a color change if the probes have hybridized to the amplified DNA target (FIG. 13B, left vial). When the PCR product is not complementary to the probes or the PCR amplification fails altogether, the probes adsorb to the gold nanoparticles and prevent aggregation (FIG. 13B, right vial).

### Example 9

### Sequence Detection and Single Base-Pair Mismatch Detection of PCR-Amplified Target cDNA

For sequence detection, 8 μL of unmodified PCR product was mixed with 6 μL of 1 μM probe solution containing either two complementary probes or two non-complementary probes in 10 mM PBS containing 0.3 M NaCl. After 5 minute denaturation at 95° C. and 1 minute annealing at 50° C., 60 μL gold colloid was added and photographs were taken. The probe sequences are as follows:
5′-CCT GTC TAA CAC CAC AG-3′ (SEQ ID NO: 14) and 5′-CCA CAG CTT GGT CAG AA-3′ (SEQ ID NO: 15) (complementary probes);
5′-ACC ACA CAC TGT CTC TC-3′ (SEQ ID NO: 16) and 5′-CTG AGC ACA CTC AGT AC-3′ (SEQ ID NO: 16) (non-complementary probes).

For single base-pair mismatch (SNP) detection, 8 μL PCR product was mixed with 6 μL of 1 μM probes overlapping the single-base mismatch, and 8 μL PCR product with 6 μL of 1 μM probes not overlapping the single base pair mismatch, respectively. The mixtures were heated at 95° C. for 5 minutes and annealed at 50° C., 54° C., and 58° C. for 1 minute, respectively, then 60 μL of gold colloid was added and a photograph was taken. The probes were as follows:
5′-CGG GAG ATG CAG GAG-3′ (SEQ ID NO: 17) and 5′-ACG GCA AGC TGG AGG-3′ (SEQ ID NO: 18) (no overlapped with SNP),
5′-CTT GCC GTC ACC GCT-3′ (SEQ ID NO: 19) and 5′-CAG CGG TGA CGG CAA-3′ (SEQ ID NO: 20) (overlapped with SNP).

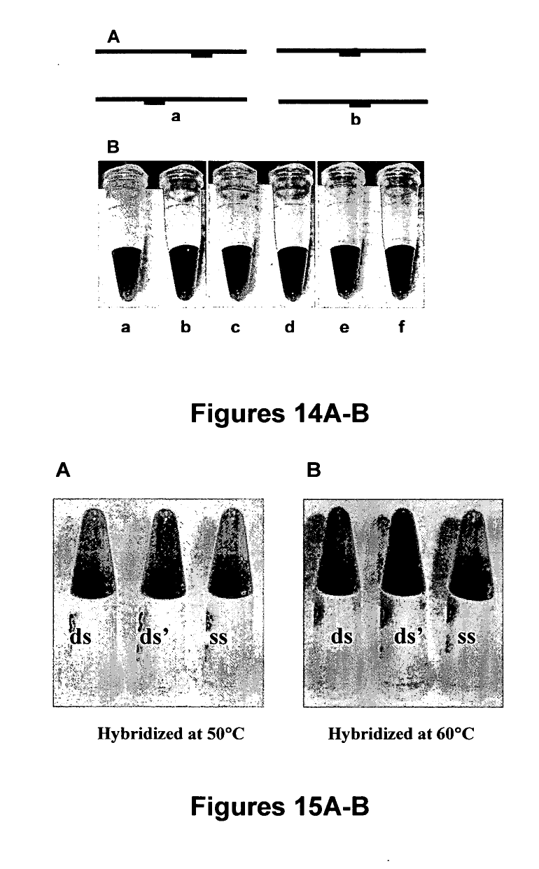

Single base-pair mismatch detection requires a slightly different protocol since a single base mismatch still permits hybridization of the probe to the target sequence. The same concept as for specific sequence detection with the strategy depicted in FIG. 14A was used. Four probes were selected that have the same melting temperature, lower than that of the PCR primers. The sequences were chosen to be complementary to the wild type sequence of the target. Two of the probes bound overlapping the position of the possible point mutation while two were used as controls and bound at locations that do not overlap the SNP under study. If a mutation exists on the target sequence, the probes covering the mutation will dehybridize at lower temperature than the control probes situated elsewhere in the sequence that are designed to be perfectly matched. At a temperature below the melting point of either sequence, the probes remain attached to the PCR amplified DNA and cannot prevent salt-induced gold nanoparticle aggregation (FIG. 14B: a, b). Above the melting temperature of both perfect and mismatched sequences, dehybridization occurs for either and gold nanoparticle aggregation is prevented (FIG. 14B: e, f). At temperatures above where the mismatched sequence dehybridizes but below where the perfectly matched sequence dehybridizes, color differences indicating the presence of a SNP are detected (FIG. 14B: c, d).

It has been demonstrated by these experiments that ss-DNA adsorbs to gold nanoparticle with a rate that is length and temperature dependent. In addition, adsorption of ss-DNA on gold nanoparticle can effectively stabilize the colloid against salt-induced aggregation. These observations were utilized to design a simple, fast colorimetric assay for PCR amplified DNA based strictly on the electrostatic properties of DNA. The approach obviates the need for gel electrophoresis and other complex sequencing procedures. It can be implemented with inexpensive commercially available materials in less than 10 minutes and no instrumentation beyond the programmable thermal cycler used for PCR is required. An important feature of the method is that, unlike chip-based assays (Fodor et al., Nature 364:555-556 (1993); Chee et al., Science 274:610-614 (1996), which are hereby incorporated by reference in their entirety) or other approaches that utilize functionalized nanoparticles (Elghanian et al., Science 277:1078-1081 (1997); Taton et al., Science 289:1757-1760 (2000); Park et al., Science 295:1503-1506 (2002); Cao et al., Science 297:1536-1540 (2002); Maxwell et al., J. Am. Chem. Soc. 124:9606-9612 (2002); Dubertret et al., Nat Biotech 19:365-370 (2001); Sato et al., J. Am. Chem. Soc. 125:8102-8103 (2003), which are hereby incorporated by reference in their entirety), hybridization occurs under optimized conditions that can be regulated independent of the assay. The assay has also been applied to clinical samples of genomic DNA that screen for SNPs associated with a hereditary cardiac arrhythmia known as long QT syndrome. It is believed that this approach can replace some traditional analytical methods for post-processing of PCR amplified DNA and that it will find broad application.

### Example 10

### RNA Detection using Modified RNA Probe

For sequence detection, 2.4 μL of 100 μM 2′-o-methyl RNA probe was mixed with 2,4 μL of 100 μM RNA target containing either one complementary probe or one non-complementary probe in 10 mM PBS and 0.3M NaCl solution. After 2 minute denaturation at 95° C. and 30 minute annealing at a temperature below the melting temperature of probe, 200 μL gold colloid was added and photographs were taken. The amount of RNA and gold colloid can be increased or decreased accordingly. In this case, a relatively large amount RNA and gold was used to measure visible spectra on regular spectrometer.

The probe and target sequences are as follows:

(SEQ ID NO: 21)

| | |
|---|---|
| 2′-o-methyl RNA probe: | AGGAAUUCCAUAGCU; |

(SEQ ID NO: 22)

| | |
|---|---|
| perfect matched target: | AGCUAUGGAAUUCCU; |
| and | |

(SEQ ID NO: 23)

| | |
|---|---|
| non-complementary target: | CGAUCACGAGAUCGA. |

For single base-pair mismatch (SNP) detection, 2.4 μL of 100 μM 2′-o-methyl RNA probe 1 (perfectly matching with target) was mixed with 2.4 μL of 100 μM targets and 2.4 μL of 100 μM 2′-o-methyl RNA probe 2 (single mismatch with target) with 2.4 μL of 100 μM target respectively. The mixtures were heated at 95° C. for 2 minutes and annealed at 50° C. and 60° C. for 30 minutes, respectively, then 200 μL of gold colloid was added and a photograph was taken.

The probe and target sequences are as follows:

(SEQ ID NO: 21)

| | |
|---|---|
| 2′-o-methyl RNA probe: | AGGAAUUCCAUAGCU; |

(SEQ ID NO: 22)

| | |
|---|---|
| perfect matched target: | AGCUAUGGAAUUCCU; |
| and | |

(SEQ ID NO: 24)

| | |
|---|---|
| single mismatched target: | AGCUAUAGAAUUCCU. |

As shown in FIGS. 15A-B, an RNA probe can be used to effectively discriminate between a SNP and a wild-type sequence.

### Example 11

### Immuno-PCR Protocol

A detection protocol employing a capture antibody and a biotinylated detection antibody coupled via streptavidin to a biotinylated DNA molecule can be employed in detecting the presence of an antigen using standard immuno-PCR procedures. If the antigen is present, PCR will result in amplification of the biotinylated DNA molecule. Assuming the antigen was present, the amplified PCR product will be detected by colorimetric or fluorimetric detection methods described in the above examples.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method for detecting presence or absence of a target nucleic acid in a test solution comprising:
combining at least one single-stranded oligonucleotide probe with a test solution potentially including a target nucleic acid to form a hybridization solution, wherein the at least one single-stranded oligonucleotide probe and the test solution are combined under conditions effective to allow formation of a hybridization complex between the at least one single-stranded oligonucleotide probe and any target nucleic acid present in the test solution;
exposing the hybridization solution to a plurality of metal nanoparticles under conditions effective to allow the at least one single-stranded oligonucleotide probe that remains unhybridized after said combining to associate electrostatically with the plurality of metal nanoparticles; and
determining whether the at least one single-stranded oligonucleotide probe has hybridized to target nucleic acid or electrostatically associated with one or more of the plurality of metal nanoparticles, wherein hybridization to the target nucleic acid or electrostatic association with one or more metal nanoparticles is indicated by an optical property of the hybridization solution.

2. The method according to claim 1, wherein exposing comprises:
contacting the hybridization solution with a suspension comprising the plurality of metal nanoparticles, and
adding a salt solution.

3. The method according to claim 2 wherein the plurality of metal nanoparticles are introduced into the hybridization solution at a concentration of between about 1 and about 100 nM.

4. The method according to claim 2 wherein the salt solution comprises a Na⁺ concentration of between about 0.01 and about 5 M.

5. The method according to claim 2 wherein said contacting and adding are carried out simultaneously.

6. The method according to claim 2 wherein said contacting and adding are carried out in succession.

7. The method according to claim 6 further comprising a delay between said contacting and adding steps.

8. The method according to claim 1 wherein at least one single-stranded oligonucleotide probe is present in the hybridization solution at a concentration of about 1 to about 10,000 nM.

9. The method according to claim 1 wherein said determining comprises:
detecting a color change of the hybridization solution after said exposing, whereby a color change indicates substantial aggregation of the plurality of metal nanoparticles in the presence of a target nucleic acid.

10. The method according to claim 7 wherein said detecting comprises comparing the color of the hybridization solution to one or more precalibrated control solutions.

11. The method according to claim 7 wherein said detecting is carried out by naked eye observation of a user.

12. The method according to claim 1 wherein the at least one single-stranded oligonucleotide probe is coupled to a fluorescent label.

13. The method according to claim 12 wherein the fluorescent label is selected from the group consisting of an organic dye, semiconductor quantum dots, lanthanide atom-containing complexes, and a fluorescent protein.

14. The method according to claim 12 wherein the optical property is a fluorescence spectra or the magnitude of a fluorescence peak.

15. The method according to claim 12 wherein said determining comprises:
detecting a substantial reduction in fluorescence caused by the fluorescent label after said exposing, which indicates quenching of fluorescence by probes electrostatically associated with one or more of the plurality of metal nanoparticles, thereby indicating the absence of a target nucleic acid.

16. The method according to claim 12 wherein the test solution comprises a plurality of different target nucleic acids and the at least one oligonucleotide probe comprises a corresponding plurality of different oligonucleotide probes, each of the plurality of different oligonucleotide probes having a distinct fluorescent label.

17. The method according to claim 1 wherein the plurality of metal nanoparticles are selected from the group consisting of gold nanoparticles, silver nanoparticles, platinum nanoparticles, mixed metal nanoparticles, and combinations thereof.

18. The method according to claim 1 wherein hybridization complexes in the hybridization solution do not significantly associate electrostatically with the plurality of metal nanoparticles.

19. The method according to claim 1 wherein the target nucleic acid is DNA.

20. The method according to claim 1 wherein the target nucleic acid is RNA.

21 The method according to claim 1 wherein the target nucleic acid is obtained from a human, a virus, a bacteria, an animal, an insect, or a plant.

22. The method according to claim 1 wherein target nucleic acid is synthetic, natural, or structurally modified DNA or RNA.

23. The method according to claim 1 wherein the target nucleic acid comprises a nucleic acid molecule coupled to a protein or polypeptide.

24. The method according to claim 1 wherein the target nucleic acid is a product of a polymerase chain reaction.

25. The method according to claim 24 wherein the test solution is obtained from a polymerase chain reaction solution, said method further comprising:
denaturing the product of the polymerase chain reaction prior to said combining.

26. The method according to claim 1 wherein each of the at least one oligonucleotide probes is between about 10 and about 50 nucleotides in length.

27. The method according to claim 1 wherein at least one oligonucleotide probe comprises structurally modified DNA or RNA.

28. The method according to claim 1 wherein the at least one oligonucleotide probe comprises a nucleotide sequence that is 100 percent complementary to the target nucleic acid.

29. A method of quantifying the amount of a target nucleic acid is a test solution, said method comprising:
providing first and second control solutions each containing known but different amounts of a target nucleic acid;
performing the method according to claim 1 on the first and second control solutions and the test solution, said performing comprising measuring the optical property of the hybridization solutions formed using the test solution and the first and second control solutions; and
calculating the quantity of the target nucleic acid in the test solution relative to the quantity of the target nucleic acid present in the first and second control solutions.

30. The method according to claim 29 wherein the optical property is the color of the hybridization solution.

31. The method according to claim 29 wherein the at least one oligonucleotide probe each comprises a fluorescent label and the optical property is a fluorescence spectra or the magnitude of a fluorescence peak caused by the fluorescent label.

32. A method of quantifying the amount of a target nucleic acid is a test solution, said method comprising:
performing the method according to claim 1 on the test solution, said performing comprising measuring the optical property of the hybridization solutions formed using the test solution; and
determining the quantity of the target nucleic acid in the test solution using the measured optical property and a calibration curve of measured optical property versus quantity of target nucleic acid.

33. A method for detecting a single nucleotide polymorphism in a target nucleic acid molecule, said method comprising:
combining (i) a test solution comprising a target nucleic acid molecule and (ii) at least one first single-stranded oligonucleotide probe comprising a nucleotide sequence that hybridizes to a region of the target nucleic acid molecule that may contain a single-nucleotide polymorphism, to form a test hybridization solution, wherein said combining is carried out under conditions effective to allow hybridization between the target nucleic acid molecule and the at least one first single-stranded oligonucleotide probe to form at least one hybridization complex;
combining (i) a control solution comprising the target nucleic acid molecule and (ii) at least one second single-stranded oligonucleotide probe comprising a nucleotide sequence that hybridizes perfectly to a region of the target nucleic acid molecule that does not contain a single-nucleotide polymorphism, to form a control hybridization solution, wherein said combining is carried out under conditions effective to allow hybridization between the target nucleic acid molecule and the at least one second single-stranded oligonucleotide probe to form at least one hybridization complex;
exposing the test and control hybridization solutions, while maintaining the hybridization solutions at a temperature that is between the melting temperature of the at least one first single-stranded oligonucleotide probe and the melting temperature of the at least one second single-stranded oligonucleotide probe, to a plurality of metal nanoparticles under conditions effective to allow unhybridized probes in the hybridization solutions to electrostatically associate with the metal nanoparticles; and
determining whether an optical property of the test and control hybridization solutions are substantially different, indicating the presence of the single nucleotide polymorphism in the target nucleic acid molecule.

34. The method according to claim 33 wherein exposing comprises:
contacting each of the test and control hybridization solutions with a plurality of metal nanoparticles, and
adding a salt solution to each of the test and control hybridization solutions.

35. The method according to claim 34 wherein the plurality of metal nanoparticles are introduced into each of the test and control hybridization solutions at a concentration of between about 1 and about 100 nM.

36. The method according to claim 34 wherein the salt solution comprises a Na⁺ concentration of between about 0.01 and about 5 M.

37. The method according to claim 34 wherein said contacting and adding are carried out simultaneously.

38. The method according to claim 34 wherein said contacting and adding are carried out in succession.

39. The method according to claim 38 further comprising a delay between said contacting and adding steps.

40. The method according to claim 33 wherein at least one first and second single-stranded oligonucleotide probes are present in the test and control hybridization solutions at a concentration of about 1 to about 100,000 nM/ml.

41. The method according to claim 33 wherein the optical property of the test and control hybridization solutions is their color, whereby a difference in color indicates presence of the single nucleotide polymorphism in the test solution.

42. The method according to claim 41 wherein said detecting is carried out by naked eye observation of a user.

43. The method according to claim 33 wherein the first and second single-stranded oligonucleotide probes comprise the same nucleotide sequence.

44. The method according to claim 33 wherein the first and second single-stranded oligonucleotide probes comprise different nucleotide sequences.

45. The method according to claim 33 wherein the plurality of metal nanoparticles are selected from the group consisting of gold nanoparticles, silver nanoparticles, platinum nanoparticles, mixed metal nanoparticles, and combinations thereof.

46. The method according to claim 33 wherein the target nucleic acid is DNA.

47. The method according to claim 33 wherein the target nucleic acid is RNA.

48 The method according to claim 33 wherein the target nucleic acid is obtained from a human, a virus, a bacteria, an animal, an insect, or a plant.

49. The method according to claim 33 wherein the target nucleic acid is a single-stranded product of a polymerase chain reaction.

50. The method according to claim 33 wherein the test solution is obtained from a polymerase chain reaction and is exposed to denaturing conditions prior to said combining.

51. The method according to claim 33 wherein each of the first and second single-stranded oligonucleotide probes is between about 10 and about 50 nucleotides in length.

52. The method according to claim 33 wherein the at least one first single-stranded oligonucleotide probe and the at least one second single-stranded oligonucleotide probe are substantially the same length.

53. The method according to claim 33 wherein one or both of the first and second single-stranded oligonucleotide probes comprises structurally modified DNA or RNA.

54. A method for detecting a single nucleotide polymorphism in a target nucleic acid molecule, said method comprising:
combining (i) a solution comprising a target nucleic acid molecule and (ii) at least one first single-stranded oligonucleotide probe comprising a nucleotide sequence and a fluorescent label attached thereto, wherein the nucleotide sequence hybridizes to a region of the target nucleic acid molecule that may contain a single-nucleotide polymorphism, to form a hybridization solution, wherein said combining is carried out under conditions effective to allow hybridization between the target nucleic acid molecule and the at least one first single-stranded oligonucleotide probe to form at least one hybridization complex;
exposing the hybridization solution to a plurality of metal nanoparticles under conditions effective to allow unhybridized probes in the hybridization solution to electrostatically associate with the metal nanoparticles;
determining a temperature of the hybridization solution where quenching of the photoluminescence by the fluorescent label begins, said temperature representing the melting temperature; and
comparing the melting temperature for the hybridization solution with a known melting temperature of a perfectly complementary probe,
wherein a difference between the melting temperatures indicates the presence of the single nucleotide polymorphism in the target nucleic acid molecule.

55. The method according to claim 54 wherein the fluorescent label is selected from the group consisting of an organic dye, semiconductor quantum dots, lanthanide atom-containing complexes, and a fluorescent protein.

56. The method according to claim 54 wherein the plurality of metal nanoparticles are introduced into the hybridization solution at a concentration of between about 1 and about 100 nM.

57. The method according to claim 54 wherein at least one single-stranded oligonucleotide probe is present in the hybridization solution at a concentration of about 1 to about 100,000 nM/ml.

58. The method according to claim 54 wherein the plurality of metal nanoparticles are selected from the group consisting of gold nanoparticles, silver nanoparticles, platinum nanoparticles, mixed metal nanoparticles, and combinations thereof.

59. The method according to claim 54 wherein the target nucleic acid is DNA.

60. The method according to claim 54 wherein the target nucleic acid is RNA.

61 The method according to claim 54 wherein the target nucleic acid is obtained from a human, a virus, a bacteria, an animal, an insect, or a plant.

62. The method according to claim 54 wherein the target nucleic acid is a single-stranded product of a polymerase chain reaction.

63. The method according to claim 54 wherein the test solution is obtained from a polymerase chain reaction and is exposed to denaturing conditions prior to said combining.

64. The method according to claim 54 wherein the single-stranded oligonucleotide probe is between about 10 and about 50 nucleotides in length.

65. The method according to claim 54 wherein the single-stranded oligonucleotide probes comprises structurally modified DNA or RNA.

66. A kit comprising:
a first container that contains a colloidal solution comprising metal nanoparticles;
a second container that contains an aqueous solution comprising at least one single-stranded oligonucleotide probe comprising a nucleotide sequence that is substantially complementary to a target nucleic acid molecule.

67. The kit according to claim 66 wherein the metal nanoparticles are characterized by a surface that is substantially free of conjugated nucleic acid molecules.

68. The kit according to claim 66 wherein the metal nanoparticles are between about 5 and about 500 nm in diameter.

69. The kit according to claim 66 wherein the metal nanoparticles are selected from the group consisting of gold nanoparticles, silver nanoparticles, platinum nanoparticles, mixed metal nanoparticles, and combinations thereof.

70. The kit according to claim 66 further comprising a containing that contains a salt solution comprises a Na+concentration of between about 0.01 and about 5 M.

71. The kit according to claim 66 wherein at least one single-stranded oligonucleotide probe is present in the aqueous solution at a concentration of about 1 to about 100,000 nM.

72. The kit according to claim 66 wherein the at least one single-stranded oligonucleotide probe is conjugated to a fluorescent label.

73. The kit according to claim 72 wherein the fluorescent label is selected from the group consisting of an organic dye, semiconductor quantum dots, lanthanide atom-containing complexes, and a fluorescent protein.

74. The kit according to claim 66 wherein the third container comprises at least two single-stranded oligonucleotide probes.

75. The kit according to claim 74 wherein each of the at least two single-stranded oligonucleotide probes is conjugated to a fluorescent label, the fluorescent label being different for the at least two single-stranded oligonucleotide probes.

76. The kit according to claim 66 wherein each of the at least one single-stranded oligonucleotide probes is between about 10 and about 50 nucleotides in length.

77. The kit according to claim 66 wherein at least one single-stranded oligonucleotide probe comprises structurally modified DNA or RNA.

78. The kit according to claim 66 wherein the at least one single-stranded oligonucleotide probe comprises a nucleotide sequence that is 100 percent complementary to the target nucleic acid.

79. A method for detecting a target nucleic acid in a test solution comprising:
subjecting a portion of a test solution potentially including a target nucleic acid to polymerase chain reaction and obtaining a product solution comprising single-stranded nucleic acid products of the polymerase chain reaction;
combining at least one single-stranded oligonucleotide probe with the product solution to form a hybridization solution under conditions effective to allow formation of a hybridization complex between the at least one single-stranded oligonucleotide probe and any target nucleic acid present in the product solution;
exposing the hybridization solution to a plurality of metal nanoparticles under conditions effective to allow any single-stranded nucleic acids in the hybridization solution to associate with the plurality of metal nanoparticles; and
determining whether the at least one single-stranded oligonucleotide probe has hybridized to target nucleic acid or electrostatically associated with one or more of the plurality of metal nanoparticles, wherein hybridization to the target nucleic acid or electrostatic association with one or more metal nanoparticles is indicated by an optical property of the hybridization solution.

80. The method according to claim 79 wherein the plurality of metal nanoparticles are introduced into the hybridization solution at a concentration of between about 1 and about 100 nM.

81. The method according to claim 79 wherein the plurality of metal nanoparticles are selected from the group consisting of gold nanoparticles, silver nanoparticles, platinum nanoparticles, and combinations thereof.

82. The method according to claim 79 wherein at least one single-stranded oligonucleotide probe is present in the hybridization solution at a concentration of about 1 to about 100,000 nM.

83. The method according to claim 79 wherein said determining comprises:
detecting a color change of the solution after said exposing, whereby a color change indicates substantial aggregation of the plurality of metal nanoparticles in the absence of a target nucleic acid.

84. The method according to claim 79 wherein the at least one single-stranded oligonucleotide probe is conjugated to a fluorescent label.

85. The method according to claim 84 wherein the fluorescent label is selected from the group consisting of an organic dye, semiconductor quantum dots, lanthanide atom-containing complex, and a fluorescent protein.

86. The method according to claim 79 wherein said determining comprises:
detecting photoluminescence caused by the fluorescent label after said exposing, which presence of a target nucleic acid.

87. The method according to claim 79 wherein hybridization complexes in the hybridization solution do not significantly associate electrostatically with the plurality of metal nanoparticles.

88. The method according to claim 79 wherein the target nucleic acid is cDNA and the polymerase chain reaction is a reverse transcription-polymerase chain reaction.

89. The method according to claim 79 wherein target nucleic acid is synthetic, natural, or structurally modified DNA or RNA.

90. The method according to claim 79 wherein the target nucleic acid comprises a nucleic acid molecule coupled to a protein or polypeptide.

91. The method according to claim 79 wherein the polymerase chain reaction is an immuno-polymerase chain reaction.

92. A method of detecting a pathogen in a sample comprising:
obtaining a sample that may contain nucleic acid of a pathogen; and
performing the method of claim 1, wherein said determining that the at least one single-stranded oligonucleotide probe has hybridized to the target nucleic acid indicates presence of the pathogen.

93. The method according to claim 92 wherein the nucleic acid isolated from the sample is RNA and the target nucleic acid is RNA.

94. The method according to claim 92 wherein the nucleic acid isolated from the sample is RNA and the target nucleic acid is cDNA, said method further comprising:
amplifying the isolated RNA by reverse-transcription polymerase chain reaction prior to said performing.

95. A method of genetic screening comprising:
obtaining a sample;
isolating DNA from the sample;
amplifying the DNA isolated from the sample; and
performing the method of claim 1, wherein said determining that the at least one single-stranded oligonucleotide probe has hybridized to the target nucleic acid indicates predisposition to a genetic condition, hereditary condition, or identifies an organism.

96. A method of detecting a protein in a sample comprising:
obtaining a sample;
performing an immuno-polymerase chain reaction procedure using the sample, wherein the immuno-polymerase chain reaction procedure results in amplification of a nucleic acid conjugated to a protein; and
performing the method of claim 1, wherein the nucleic acid that is conjugated to the protein is the target nucleic acid, and wherein said determining that the at least one single-stranded oligonucleotide probe has hybridized to the target nucleic acid indicates that the protein is present in the sample.

97. A method of quantifying the amount of amplified nucleic acid prepared by polymerase chain reaction, said method comprising:
providing two or more fluorescently labeled oligonucleotide primers that each comprise a nucleotide sequence capable of hybridizing to a nucleic acid molecule, or its complement, to be amplified;
performing polymerase chain reaction using a target nucleic acid molecule and/or its complement, and the provided fluorescently labeled oligonucleotide primers; and
performing the method of claim 1 on a sample obtained after said performing polymerase chain reaction, wherein the level of fluorescence detected from the sample indicates the amount of primer that has been incorporated into an amplified nucleic acid molecule.
